(19) United States
(12) Patent Application Publication
(10) Pub. No.: US 2012/0076729 A1
(43) Pub. Date: Mar. 29, 2012

(54) METHODS OF TREATING CANCERS

(75) Inventors: Dinh Le Mary Chau, Waltham, MA (US); Jesper Gromada, Concord, MA (US); Zhidan Wu, Boston, MA (US)

(73) Assignee: NOVARTIS AG, Basel (CH)

(21) Appl. No.: 13/375,556

(22) PCT Filed: Jun. 2, 2010
(86) PCT No.: PCT/EP10/57735

§ 371 Date: Dec. 1, 2011

Related U.S. Application Data
(60) Provisional application No. 61/184,156, filed on Jun. 4, 2009.

(51) Int. Cl. A61K 38/18; A61K 39/395; G01N 33/566; A61P 35/00; C12Q 1/68; G01N 33/573; A61K 49/00; A61K 31/713

(52) U.S. Cl. 424/9.1; 424/158.1; 514/44 A; 514/9.1; 435/6.12; 435/7.21

(57) ABSTRACT

The present application describes methods for treating and/or preventing cancer or a cancer symptom, e.g., AMPK-related cancers, in a patient in need thereof comprising administering to the patient a therapeutically effective amount of an FGF, e.g., FGF21, or an FGF agonist, or a pharmaceutical composition comprising the same.

### BACKGROUND OF THE INVENTION

Fibroblast growth factor 21 (FGF, e.g., an FGF21) is a member of the FGF protein family (U.S. Pat. No. 6,716,626). The FGF proteins identified to date belong to a family of signaling molecules that regulate growth and differentiation of a variety of cell types. The significance of FGF proteins to human physiology and pathology relates in part to their roles in embryogenesis, in Hood vessel de.velopment and growth, and in bone growth. The majority of FGF family members have been associated with cellular activities including mitosis, development, transformation, angiogenesis, and cell survival. Several members of the FGF family and their biological roles are described in Crossley et al., Development 121:439-451 (1995), Ohuchi et al., Development 124:2235-2244 (1997); Gemel et al., Genomics 35:253-257 (1996); and Ghosh et al., Cell Growth and Differentialion 7:1425-1434 (1996),

Fibroblast growth factor (FGF, e.g, an FGF21) has been identified as a potent metabolic regulator. Systemic administration of FGF, e.g., an FGF21 to rodents and rhesus monkeys with diet-induced or genetic obesity and diabetes exerts strong anti-hyperglycemic and triglyceride-lowering effects, and reduction of body weight. (Coskun, T, et al, (2008) Endocrinology 149:6018-6027)(Kharitonenkov,A, et al. (2005) Journal of Clinical Investigation 115:1627-1635)(Kharitonenkov,A, et al. (2007) Endocrinology 148:774-781)(Xu, J, et al. (2009) Diabetes 58:250-259).

FGF, e.g., an FGF21 binds to the FGF receptors (FGFRs), FGFR1c and FGFR4, which enables the cytoplasmic kinase domains to transphosphorylate one another, and become activated. Recent studies have shown thin β-klotho, a single-pass trans-membrane protein related to klotho, functions as a cofactor that is required for FGF, e.g., an FGF21 signaling. (Kharitonenkov,A, et al. (2008) Journal of Cellular Physiology 215:1-7)(Kurosu,H, et al. (2007) JBC 282:26687-26695)(Ogawa, Y. et al. (2007) PNAS 104:7432-7437) Because β-klotho acts as an essential cofactor for FGF, e.g., an FGF21 signaling, its expression limits the tissue-specific activities of FGF, e.g., an FGF21 to the liver, pancreas and adipose tissues, where β-klotho is predominantly expressed.

More recently, the role of FGF, e.g., an FGF21 in the liver has been established; FGF, e.g., an FGF21 has been demonstrated to play an important role in regulating energy homeostasis during starvation. (Badman, M K, et al. (2007) Cell Metabolism 5:426-437)(Galman, C, et al. (2008) Cell Metabolism 8:169-174)(Inagaki, T, et al. (2007) Cell Metabolism 5:415-425) FGF, e.g., an FGF21 is induced by fasting, and is required for activation of hepatic lipid oxidation, triglyceride clearance and ketogenesis during fasting. These findings identify FGF, e.g., an FGF21 as an important regulator of hepatic lipid homeostasis and metabolism. Despite the importance of FGF, e.g., an FGF21 as a regulator of hepatic metabolism, much less is known about the role of FGF, e.g., an FGF21 in adipose tissues.

A recent study reported FGF, e.g., an FGF21 induction of PGC-1α mRNA expression in white adipose tissue of high fat diet induced obese mice. PGC-1α stimulates mitochondrial biogenesis and respiration through induction of NRF1 and NRF2 gene expression. (Wu, Z D, et al, (1999) Cell 98:115-124) PGC-1α is induced by exercise and by pharmacological activation of AMPK in skeletal muscle. (Baar, K, et al. (2002) Faseb Journal 16:1879-1886)(Bergeron, R, et al. (2001) American Journal of Physiology-Endocrinology and Metabolism 281:E1340-E1346)(Winder, W W, et al. (2000) Journal of Applied Physiology 88:2219-2226)

AMPK is a metabolic sensor that is conserved in eukaryotes. Activation of AMPK acts to maintain cellular energy stores, switching on catabolic pathways that produce ATP, by enhancing oxidative metabolism and mitochondrial biogenesis, while turning off anabolic pathways that consume ATP. (Hardie., D G (2007) Nature Reviews Molecular Cell Biology 8:774-785) Recent evidence demonstrated the importance of AMPK in the therapeutic benefits of metformin, thiazolidinediones and exercise, which form the cornerstones of the clinical management of type 2 diabetes and associated metabolic disorders. (Shaw, R J, et al. (2005) Science 310:1642-1646)(Zhou, G C, et al, (2001) Journal of Clinical Investigation 108:1167-1174)

Recent reports have also indicated that AMPK may also be a beneficial target for cancer treatment. Cancer cells have characteristic metabolic changes from normal cells and being a key metabolic regulator, AMPK may regulate the switch. AMPK may act to inhibit tumorigenesis through regulation of cell growth, cell proliferation, autophagy, stress responses and cell polarity.

Demonstration of a link shown between Fibroblast Growth Factors, such as FGF, e.g., an FGF21, and AMPK, such as that herein, would implicate the former in cancers, particularly AMPK-related cancers. Here we demonstrate that FGF, e.g., an FGF21 regulates energy homeostasis via enhancement of mitochondrial function and efficiency through an AMPK-PGC-1α-dependent pathway, and demonstrate that among other things, FGF, e.g., FGF21, regulates mitochondrial activity and enhances oxidative capacity through an AMPK-dependent mechanism.

### SUMMARY OF THE INVENTION

In some aspects, the present invention provides compositions comprising a Fibroblast Growth Factor agonist, e.g., an FGF, e.g., an FGF21 agonist, and one or more pharmaceutically acceptable carriers.

In some aspects, the present invention provides compositions comprising an FGF (e.g., an FGF21) modulator and one or more pharmaceutically acceptable carriers, wherein the FGF modulator is an agonizing antibody (e.g., that binds an epitope in a domain of FGF, e.g., FGF21 selected from the group consisting of the signal peptide domain and the FGF receptor binding domain) a mimetic; fusion proteins; or a therapeutic FGF protein or fragment thereof,

In some aspects, the present invention provides a fragment of an FGF, e.g., an FGF21 polypeptide comprising between 10 and 209 contiguous amino acid residues of SEQ ID NO:2, wherein the fragment retains an FGF, e.g., an FGF21, polypeptide activity.

In some aspects, the present invention provides, methods of treating cancer or a cancer symptom, e.g., AMPK-related cancers, in a patient in need thereof comprising administering to the patient a therapeutically effective amount of an FGF, e.g., FGF21, or an FGF agonist.

In some aspects, the present invention provides methods of identifying a patient susceptible to FGF, e.g., an FGF21, therapy comprising (a) detecting evidence of underexpression of AMPK in a cell sample from the patient as compared to a control cell sample. The presence of evidence of underexpression of AMPK in the sample is indicative of a patient who is a candidate for FGF, e.g., an FGF21 therapy and the absence of evidence of AMPK underexpression in the sample is indicative of a patient who is not a candidate for FGF, e.g, an FGF21 therapy. Therapeutically effective amount of FGF, FGF21 agonists are administered to the patient if the patient is a candidate for FGF, e.g., an FGF21 therapy; and conventional cancer therapeutics are administered to the patient if the patient is not a candidate for FGF', e.g., an FGF21 therapy.

In further aspects, the present invention provides methods of modulating one or more activities in cells that express AMPK comprising contacting the cells with an amount of an FGF, e.g., an FGF21 agonist effective to modulate the one or more activities.

In some aspects, the present invention provides methods for detecting a tumor in a patient afflicted with an AMPK-related cancer or cancer symptoms, comprising administering to the patient a composition comprising an FGF, e.g,, an FGF21 agonist linked to an imaging agent and detecting the localization of the imaging agent in the patient.

In some aspects, the present invention provides methods of identifying an FGF21agonist capable of AMPK-related cancer inhibition. The methods comprise contacting a cell expressing AMPK with a candidate FGF21 agonist and determining whether AMPK levels are elevated, and/or whether AMPK is activated, relative to a control. Elevation of AMPK levels and/or activation of AMPK is indicative of a cancer inhibitor,

In some aspects, the present invention provides methods a method of assessing whether a subject afflicted with an AMPK-related cancer or cancer symptoms would be susceptible to treatment by FGF21 or FGF21 agonist administration comprising measuring AMPK or SIRT1 levels in a sample from said subject as compared to a control sample, wherein lower AMPK or SIRT1 levels in the subject sample compared to the control indicate that said subject would be amenable to treatment by FGF21 or FGF21 agonist administration,

In some aspects, the present invention provides methods of detecting a tumor in a patient comprising administering, to the patient a composition comprising FGF21 or an FGF21 agonist linked to an imaging agent and detecting the localization of the imaging agent in the patient.

In some aspects, the present invention provides methods of delivering a cytotoxic agent or a diagnostic agent to one or more cells that express AMPK. The methods comprise (a) providing the cytotoxic agent or the diagnostic agent conjugated to an anti-FGF, e.g., an FGF21 agonist; and (b) exposing the cell to the antibody-agent or fragment-agent conjugate.

In further aspects, the present invention provides methods for determining the effectiveness of a candidate FGF, e.g., an FGF21 agonist. The methods comprise contacting AMPK-expressing cells with the candidate FGF, e.g., an FGF21 agonist and determining whether a downstream marker of AMPK is modulated. Modulation of the downstream marker indicates that the candidate FGF, e.g., an FGF21 agonist is an effective anti-cancer medication.

In further aspects, the present invention provides methods of determining whether a cancer is susceptible to FGF, e,g.,an FGF21-related treatment comprising comparing AMPK expression in cancer and control cells. Downregtdation of AMPK expression in the cancer cells as compared to the control cells indicates that the cancer is susceptible to an FGF, e.g., an FGF21-related treatment.

In further aspects, the present invention provides methods of determining whether a cancer is susceptible to an FGF, e.g., an FGF21-related treatment comprising contacting a cancer sample with an FGF, e.g, an FGF21 agonist, and measuring an AMPK downstream marker in the cancer sample. Modulation of the downstream marker in the presence of the agonist as compared to the downstream marker in the absence of the agonist indicates that the cancer is susceptible to an FGF, e.g., an FGF21-related treatment.

In some aspects, the present invention provides methods of treating a cancer patient comprising determining whether a cancer is susceptible to an FGF, e.g., an FGF21-related treatment and administering to the patient an FGF, e.g., an FGF21 agonist if the patient has an cancer type which is susceptible to an FGF, e.g., an FGF21-related treatment, or administering to the patient a conventional cancer therapeutic if the patient does not have a cancer type which is susceptible to an FGF, e.g., an FGF21-related treatment.

These and other aspects of the present invention will be elucidated in the following detailed description of the invention.

### BRIEF DESCRIPTION OF THE DRAWINGS

FIG. 1: Effects of FGF21 on 3T3-L1 gene expression. Data shown are from quantitative RT-PCRs, normalized to the housekeeping gene, TATA binding protein (Tbp), 3T3-L1 adipocytes were treated with PBS control (gray bars) or FGF21 (black bars) for 72 hours. n=3, **p<0.01 and *p<0.05.

FIG. 2: FGF21 enhances mitochondrial function. (A) Citrate synthase activity in 72 hour treated 3T3-L1 adipocytes. (B) Oxygen consumption in 3T3-L1 adipocytes treated with PBS (gray bars) or FGF21 (black bars). Y axis shows OCR (pMoles/min) ***p<0.001 and *p<0.05.

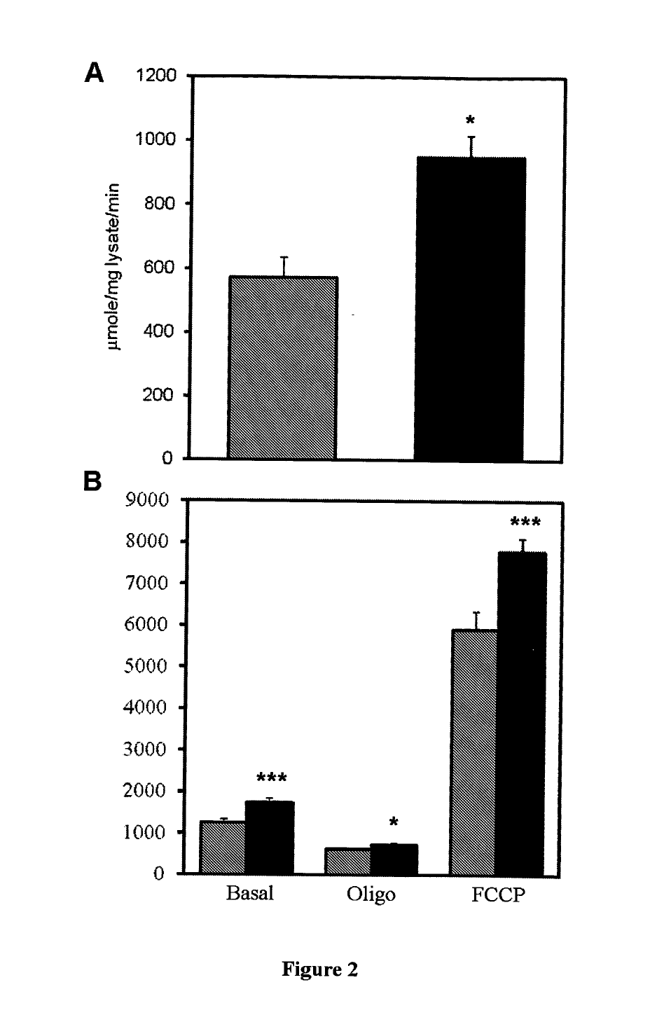

FIG. 3: FGF21 effects in adipocytes require AMPK activity. (A) 3T3-L1 adipocytes were treated with DMSO or Compound C for 20 hours and PBS (gray bars) or FGF21 (black bars) for 72 hours. Data shown are from quantitative RT-PCRs, normalized to the housekeeping gene, TATA binding protein (Tbp) Relative mRNA levels are shown on the Y axis. (B) Western blot of phosphorylated AMPK in 3T3-L1 adipocytes treated with FGF21. (C) Basal oxygen consumption in 3T3-L1 adipocytes treated with DMSO or Compound C and PBS (gray) or FGF21 (black). **p<0.01 and ***p<0.0001.

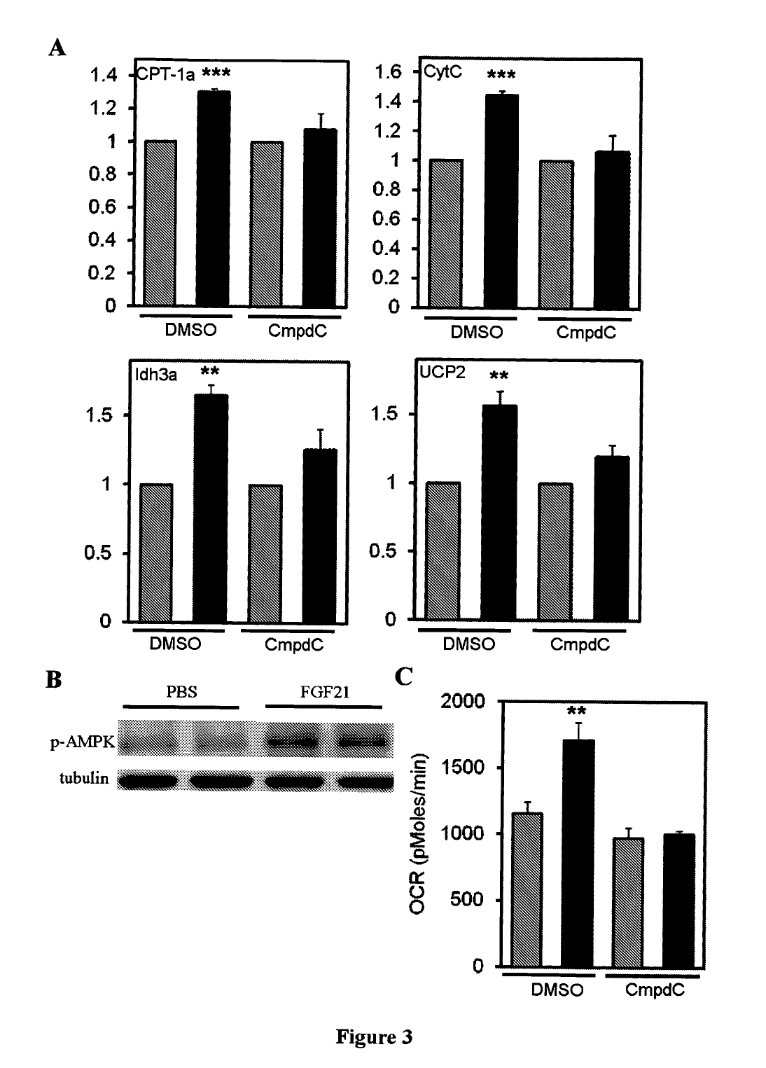

FIG. 4: Effect of FGF21 on gene expression in ob/ob WAT. (A) Data shown are from quantitative RT-PCRs, normalized to the housekeeping gene. TATA binding protein (Tbp). Male C57B⅙ mice (n=8/group) were treated with vehicle control (PBS; gray bars) or 60 mg FGF21 (black bars). (B) Western blot of p-AMPK. in WAT. (C) Quantification of the levels of p-AMPK in WAT. **p<0.01 and *p<0.05.

FIG. 5: FGF21 effects in adipocytes require PGC-1α and β. 3T3-L1 adipocytes were infected with shRNA-CMV control, shRNA-PGC-1 α or shRNA-PGC-1 β adenovirus and treated with PBS (gray bars) or FGF21 (black bars) for 72 hours. Data shown are from quantitative RT-PCRs, normalized to the housekeeping gene, TATA binding protein (Tbp). ***p<0.0001, **p<0.01 and *p<0.05.

FIG. 6: (A) Blood glucose levels in DIO mice. (B) Plasma glucose levels during OGTT. (C) Glucose AUC, above basal during OGTT. Gray bars: PBS; black bars: FGF21. *p-value<0.05.

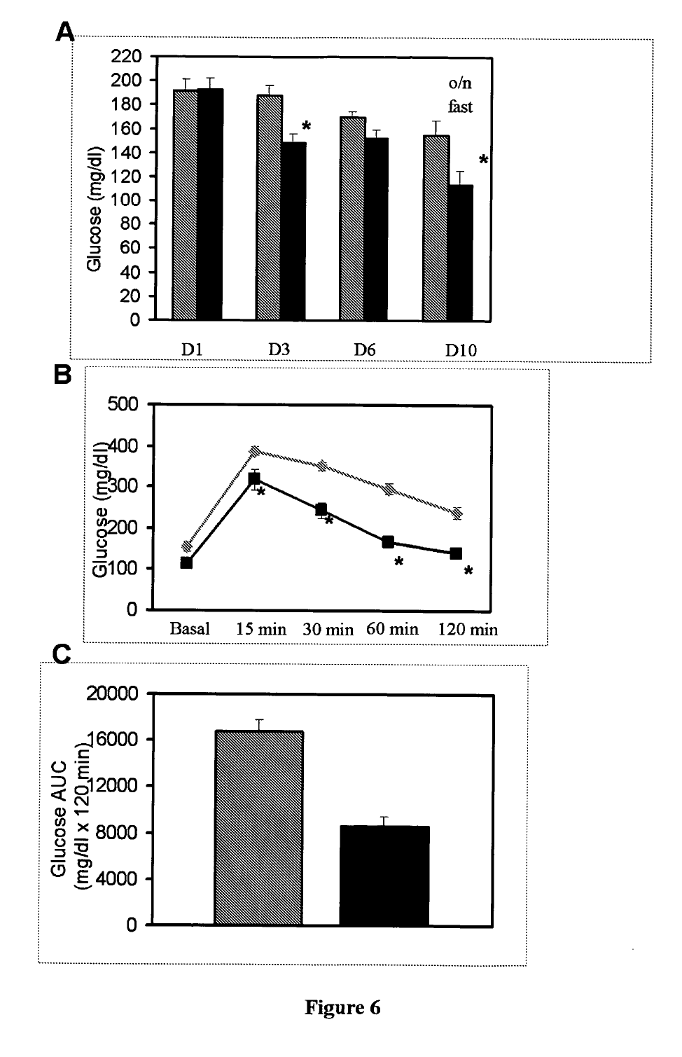

FIG. 7: (A) Body weight in DIO mice. The data points correspond to days 1, 3, 6, 9, 10, and 14. (B) Epididymal fat mass in DIO mice. (C) Daily food intake. Gray bars: PBS; black bars: FGF21, The data points correspond to days 1, 3, 6, and 10, *p-value<0.05.

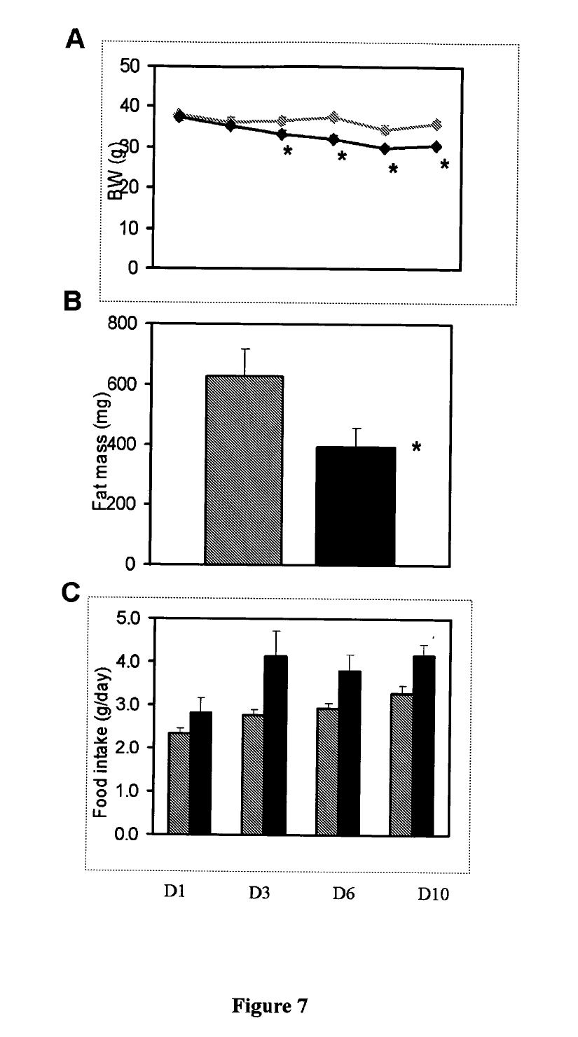

### DETAILED DESCRIPTION OF THE INVENTION

Fibroblast growth factor 21 (FGF21) has been identified as a potent metabolic regulator. A link is demonstrated herein for the first time between Fibroblast Growth Factors, such as FGF, e.g., an FGF21, and AMPK, thereby implicatating, FGFs in cancers, particularly AMPK-related cancers. FGF, e.g., FGF21, is show herein to regulate energy homeostasis via enhancement of mitochondrial function and efficiency through an AMPK- and SIRT-PGC-1α-dependent pathway. FGF, e.g., FGF21, regulates mitochondrial activity and enhances oxidative capacity through an AMPK-dependent mechanism.

Despite recent evidence implicating FGF21 in the regulation of glucose, lipid and energy homeostasis, the mechanisms via which FGF21 acts as a metabolic regulator remain largely unknown. The present application demonstrates for the first time that FGF21 regulates energy homeostasis via enhancement of mitochondrial function and efficiency through activation of AMPK and SIRT1 in vitro and in vivo. FGF21 increases the phosphorylation of AMPK. Activation of AMPK leads to increased cellular NAW levels, which in turn activate SIRT1 and subsequently impacts multiple metabolic pathways. The effect of FGF21 in enhancing mitochondrial function is mediated through PGC-1α.

The activation of AMPK and SIRT1 converge on enhanced mitochondrial oxidative function as demonstrated by significant increases in oxygen consumption, citrate synthase activity and induction of key metabolic genes, including CPT-1a and CytC, in FGF21-treated adipocytes. Inhibition of AMPK and SIRT1 activities by dominant-negative or shRNA adenovirus attenuated the effects of FGF21 on oxygen consumption and gene expression, suggesting that FGF21 regulates mitochondrial activity and enhances oxidative capacity through an AMPK and SIRT1-dependent mechanism. The effects of FGF21 on AMPK and SIRT1 are observed both in vitro and in vivo, suggesting that these effects are likely to be direct and not secondary to the changes in body weight and adiposity.

The present studies demonstrate that the effects of FGF21 on mitochondrial function require PGC-1α, and further show that FGF21 induces AMPK and SIRT1 activities, which act in concert with PGC-1α to regulate energy homeostasis,

Treatment of ob/ob mice with FGF21 elicits similar beneficial metabolic effects to those observed in transgenic mice that overexpress SIRT1. Both FGF21-treated and SIRT1 transgenic animals exhibit reductions in body weight and plasma insulin and glucose levels, as well as improved glucose tolerance. (Bordone, L., et al, (2007) Aging Cell 6, 759-767). Additionally, treatment of animals with AMPK activators, such as metformin, induce similar metabolic effects to FGF21, including glucose lowering. The convergent biological effects observed in FGF21-treated animals with SIRT1 and AMPK activation further support our hypothesis that FGF21 induces AMPK and SIRT1 activities.

A possible mechanism of FGF21 activation of AMPK is through ERK ½ and LKB1 interaction. FGF21 signals through β-klotho and the FGF receptors results in activation of ERK ½.

The actions of FGF21 are limited to tissues that express β-klotho, including the liver, pancreas and adipose tissues. The studies described herein show that FGF21 stimulates AMPK and SIRT1 activities in adipose tissue. Recently, FGF21 was found to increase PGC-1α expression, fatty acid oxidation and tricarboxylic acid cycle flux in the liver. (Potthoff, M. J., et al. (2009) PNAS 106, 10853-10858.) FGF21 has been shown previously to protect pancreatic cells from glucolipotoxicity-induced apoptosis and dysfunction. (Wente. W., et al. (2006) Diabetes 55, 2470-2478). SIRT1 and NAD metabolism have been implicated to be critical to pancreatic β-cell function and health (Ramsey, K. et al. (2009) Aging Cell 7, 78-88); it would be of interest to determine whether the effects of FGF21 on β-cell survival and function are mediated through SIRT1. It is likely that the effects of FGF21 on these pathways occur in other tissues, such as the liver and pancreas, in addition to adipose tissue, which collectively contribute to the improvement of energy homeostasis.

Taken together, the results described herein reveal a scenario in which FGF21 enhances mitochondrial function and oxidative capacity via activation of AMPK and SIRT1 and their downstream targets, including PGC-1α, in adipocytes. Consequently, the physiologic integrative response to these changes in energy production and fatty acid oxidation improves lipid profiles and insulin sensitivity, accounting for the beneficial metabolic effects of FGF21. Furthermore, the data in human adipocytes demonstrate that FGF21 induces similar effects on mitochondrial function in both rodents and humans.

### Definitions

Various definitions are used throughout this document. Most words have the meaning that would be attributed to those words by one skilled in the art. Words specifically defined either below or elsewhere in this document have the meaning provided in the context of the present invention as a whole and as are typically understood by those skilled in the art.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick. and N. Kaplan, eds., Academic Press, Inc.); and Handbook of Experimental Immunology, Vols. I-IV (D. M. Weir and C. C. Blackwell, eds., 1986, Blackwell Scientific Publications); and Sambrook et al., Molecular Cloning: A Laboratory Manual (2nd Edition, 1989).

As used herein, the singular forms “a,” “an” and “the” include plural references unless the content clearly dictates otherwise. Thus, for example, reference to “an antibody” includes a mixture of two or more such antibodies.

As used herein, the term “about” refers to +/−20%, +/−10%, or +/−5% of a value.

As used herein, the term “FGF, e.g, an FGF21” refers to a member of the fibroblast growth factor (FGF) protein family. An mRNA sequence of FGF21 (GenBank Accession No. NM_{—}019113) is set forth as SEQ ID NO:1, and an amino acid sequence of FGF21 (GenBank Accession No. NP_{—}061986) is set forth as SEQ ID NO:2.

As used herein, the term “FGF, e.g., an FGF21 receptor” refers to a membrane-bound receptor for FGF, e.g., an FGF21, Binding of an FGF, e.g., an FGF21 to an FGF receptor results in a cellular response and/or activity, such as a cell signaling event, In some embodiments the FGF receptor is FGFR-1 or FGFR-2.

The terms “polypeptide” and “protein”, are used interchangeably and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “individual”, “subject”, “host” and “patient” are used interchangeably and refer to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, “cancer” refers to primary or metastatic cancers, leukemias, or lymphomas. The term “cancer cells” refers to cells that are transformed. These cells can be isolated from a patient who has cancer, or be cells that are transformed in vitro to become cancerous. Cancer cells can be derived from many types of samples including any tissue or cell culture line. In some embodiments the cancer cells are hyperplasias, tumor cells, or neoplasms. In some embodiments, the cancer cells are isolated from colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma and melanoma. In some embodiments, the cancer cells are taken from established cell lines that are publicly available. In some embodiments, cancer cells are isolated from pre-existing patient samples or from libraries comprising cancer cells. In some embodiments, cancer cells are isolated and then implanted in a different host, e.g., in a xenograft. In some embodiments cancer cells are transplanted and used in a SCID mouse model. In some embodiments, the cancer is colon cancer. As used herein, the term “colon cancer” is used interchangeably with “rectal cancer” and “colorectal cancer,” and refers to a cancer that originates in the colon or rectum,

As used herein, “AMPK” means AMP-activated protein kinase. “AMPK -related cancers” are those characterized by, for example, low levels of AMPK protein, and/or downregulation of the gene(s) encoding AMPK. Such downregulation can be abrogated or ameliorated by the administration of the FGF, e.g., an FGF21 agonists of the invention, thereby activating AMPK. Examples of AMPK-related cancers include but are not limited to breast cancer, prostate cancer, lung cancer, pancreatic and biliary cancers, malignant melanomas, carcinomas (e.g., hepatocellular carcinomas), and colon cancer.

As used herein, the term “transformed” refers to any alteration in the properties of a cell that is stably inherited by its progeny. In some embodiments, “transformed” refers to the change of normal cell to a cancerous cell, e.g., one that is capable of causing tumors. In some embodiments, a transformed mil is immortalized. Transformation can be caused by a number of factors, including overexpression of a receptor in the absence of receptor phosphorylation, viral infection, mutations in oncogenes and/or tumor suppressor genes, and/or any other technique that changes the growth and/or immortalization properties of a cell.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, or the like.

As used herein, the term “metastasis” refers to a cancer which has spread to a site distant from the origin of the cancer, e.g, from the primary tumor. Sites of metastasis include without limitation, the bone, lymph nodes, lung, liver, and brain.

As used herein, the term “angiogenesis” refers to the the growth of new blood vessels from pre-existing vessels.

As used herein, the term “clinical endpoint” refers to a measurable event indicative of cancer. Clinical endpoints include without limitation, time to first metastasis, time to subsequent metastasis, size and/or number of metastases, size and/or number of tumors, location of tumors, aggressiveness of tumors, quality of life, pain and the like. Those skilled in the art are credited with the ability to determine and measure clinical endpoints. Methods of measuring clinical endpoints are known to those of skill in the art.

As used herein, the term “sample” refers to biological material from a patient. The sample assayed by the present invention is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized.

As used herein, the term “biological molecule” includes, but is not limited to, polypeptides, nucleic acids, and saccharides.

As used herein, the term “modulating” refers to a change in the quality or quantity of a gene, protein, or any molecule that is inside, outside, or on the surface of a cell. The change can be an increase or decrease in expression or level of the molecule. The term “modulates” also includes changing the quality or quantity of a biological function/activity including, without limitation, cell proliferation, growth, adhesion, apoptosis, intracellular signaling, cell-to-cell signaling, and the like.

As used herein, the term “differentially expressed in a cancer cell” and “a polynucleotide that is differentially expressed in a cancer cell” are used interchangeably herein, and refer to a polynucleotide that represents or corresponds to a gene that is differentially expressed in a cancerous cell when compared with a cell of the same cell type that is not cancerous, e.g., mRNA is found at levels at least about 25%, at least about 50% to about 75%, at least about 90%, at least about 1.5-fold, at least about 2-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source, or between one cell in situ and a second cell removed from its tissue source. In some embodiments, the gene is upregulated in the cancer gene as compared to the normal cell.

As used herein, the phrase “increasing cancer cell apoptosis” refers to increasing apoptosis of cancer cells that differentially express AMPK in the presence of an FGF, e.g., an FGF21 agonist. In this context, cancer cell apoptosis can be increased by an FGF, e.g., an FGF21 agonist at least 25%, at least 50%, at least 75%, at least 85%, at least 90%, at least 95%, up to 100% relative to cancer cell apoptosis in the absence of an FGF, e.g.., an FGF21 agonist, Comparisons of cancer cell apoptosis can be accomplished by measuring, for example. DNA fragmentation, caspase activity, loss of mitochondrial membrane potential, increased production of reactive oxygen species (ROS), intracellular acidification, chromatin condensation, phosphatidyl serine (PS) levels at the cell surface, and increased cell membrane permeability.

DNA fragmentation can be measured, for example, with the TUNEL assay (terminal deoxynucleotide transferase dUTP nick end labeling). Commercial versions of the assay are widely available, for example. APO-BrdU™ TUNEL Assay Kit (Invitrogen), APO-DIRECT™ Kit (BD Biosciences Pharmingen) and ApoAlert™ DNA Fragmentation Assay Kit (Clontech, a Takara Bio Company).

Caspase activity can be monitored via fluorogenic, chromogenic and luminescent substrates specific for particular caspases. Commercial assay kits are available for at least caspases 1, 2, 3, 6, 7, 8 and 9. (See, for example, Invitrogen, Chemicon, CalBiochem, BioSource International, Biovision).

Loss of mitochondrial membrane potential can be measured with fluorescent dyes that differentially accumulate in healthy active mitochondria. One non-limiting example is the MitoTracker Red system from Invitrogen.

Production of reactive oxygen species (ROS) can be measured with fluorescent dyes including, for example, H2DCFDA (Invitrogen).

Intracellular acidification can be measured with fluorescent or chromogenic dyes.

Chromatin condensation can be measured with fluorescent dyes including, for example, Hoechst 33342.

Phosphatidyl serine (PS) levels can be measured at the cell surface. For example, Annexin V has a high affinity for PS. Numerous commercially available assays are suitable to monitor the binding of labeled AnnexinV to the cell surface.

Cell membrane permeability can be measured using dyes, such as the fluorescent dye, YO-PRO-1 (Invitrogen) which can enter apoptotic, but not necrotic cells.

As used herein, the phrase “inhibits cancer cell growth” refers to inhibition or abolition of cancer cell growth in the presence of an FGF, e.g., an FGF21 agonist wherein the cell differentially expresses AMPK. In this context, cancer cell growth can be decreased by FGF, e.g., an FGF21 agonist at least 25%, at least 50%, at least 75%, at least 85%, at least 90%, at least 95%, up to 100% relative to cancer cell growth in the absence of an FGF, e.g., an FGF21 agonist. Comparisons of cancer cell growth can be accomplished using, for example. MTT assay (for example, the Vybrant® MTT Cell Proliferation Assay Kit (Invitrogen)); BrdU incorporation (for example, the Absolute-S SBIP assay (Invitrogen)); measuring intracellular ATP levels (for example using ATPLite™-M, 1,000 Assay Kit (PerkinElmer) or ATP Cell Viability Assay Kit (BioVision)); DiOc18 assay, a membrane permeable dye (Invitrogen);Glucose-6-phosphate dehydrogenase activity assay (for example, the Vibrant cytotoxicity assay (Invitrogen)); or measuring cellular LDH activity.

As used herein, the phrase “inhibits tumor formation” refers to inhibition or abolition of tumor formation in the presence of an FGF, e.g., an FGF21 agonist wherein the tumor comprises cells that differentially express AMPK. In this context, tumor formation can be decreased by an FGF, e.g., an FGF21 agonist at least 25%, at least 50%, at least 75%, at least 85%, at least 90%, at least 95%, and up to 100% relative to tumor formation in the absence of an FGF, e.g.. an FGF21 agonist. Comparisons of tumor formation can be accomplished using, for example, cell based assays (for example colony formation in soli agar); in vivo models of tumor formation typically relying upon injecting the cells of interest into animals (for example, athymic mice or rats, irradiated mice or rats; inoculation into immunologically privileged sites such as brain, cheek pouch or eye; inoculation of syngeneic animals), and monitoring the size of the mass after a defined time period.

As used herein, the phrase “increases phosphorylation of AMPK” refers to the upregulated phosphorylation of AMPK by FGF, e.g., FGF21 agonists. In this context, AMPK phosphorylation can be increased by an FGF, e.g., an FGF21 agonist at least 25%, at least 50%, at least 75%, at least 85%, at least 90%, at least 95%, up to 100% relative to AMPK phosphorylation in the absence of an FGF, e.g., an FGF21 agonist. Phosphorylation levels can be assessed using phosphorylation assays known to those of skill in the art.

As used herein, the term “up-regulates” refers to an increase, activation or stimulation of an activity or quantity. For example, in the context of the present invention. FGF, e.g., an FGF21 agoninsts may increase the level of AMPK phosphorylation. Up-regulation may be at least 25%, at least 50%, at least 75%, at least 100%, at least 150%, at least 200%, at least 250%, at least 400%, or at least 500% as compared to a control.

As used herein, the term “don-regulates” refers to an decrease, inactivation, agbrogation, or mitigation of an activity or quantity. For example, in the context of the present. invention, AMPK is down-regulated in certain cancer types. These would be particularly amenable to treatment by the FGF, e.g., FGF21, agonists of the invention.

As used herein, the term “N-terminus” refers to at least the first 10 amino acids of a protein.

As used herein, the terms “N-terminal domain” and “N-terminal region” are used interchangeably and refer to a fragment of a protein that begins at the first amino acid of the protein and ends at any amino acid in the N-terminal half of the protein. For example, the N-terminal domain of FGF, e.g., an FGF21 is from amino acid 1 of SEQ ID NO:2 to any amino acid between about amino acids 9 and 209 of SEQ ID NO:2.

As used herein, the term “C-terminus” refers to at least the last 10 amino acids of a protein.

As used herein, the terms “C-terminal domain” and “C-terminal region” are used interchangeably and refer to a fragment of a protein that begins at any amino acid in the C-terminal half of the protein and ends at the last amino acid of the protein. For example, the C-terminal domain of FGF, e.g., an FGF21 begins at any amino acid from amino acid 105 to about amino acid 200 of SEQ ID NO:2 and ends at amino acid 209 of SEQ ID NO:2.

The term “domain” as used herein refers to a structural part of a biomolecule that contributes to a known or suspected function of the biomolecule. Domains may be co-extensive with regions or portions thereof and may also incorporate a portion of a biomolecule that is distinct from a particular region, in addition to all or part of that region.

As used herein, the term “signal domain” (also called “signal sequence” or “signal peptide”) refers to a peptide domain that resides in a continuous stretch of amino acid sequence at the N-terminal region of a precursor protein (often a membrane-bound or secreted protein) and is involved in post-translational protein transport. In many cases the signal domain is removed from the full-length protein by specialized signal peptidases after the sorting process has been completed. Each signal domain specifies a particular destination in the cell for the precursor protein. The signal domain of FGF21 is amino acids 1-28 of SEQ ID NO:2 (see GenPept Accession No NP_{—}061986).

As used herein, the term “receptor binding domain” refers to any portion or region of a protein that contacts a membrane-bound receptor protein, resulting in a cellular response, such as a signaling event.

As used herein, the term “ligand binding domain” refers to any portion or region of a protein retaining at least one qualitative binding activity of a corresponding native sequence of FGF, e.g., an FGF21.

The term “region” refers to a physically contiguous portion of the primary structure of a biomolecule. In the case of proteins, a region is defined by a contiguous portion of the amino acid sequence of that protein. In some embodiments a “region” is associated with a function of the biomolecule.

The term “fragment” as used herein refers to a physically contiguous portion of the primary structure of a biomolecule. In the case of proteins, a portion is defined by a contiguous portion of the amino acid sequence of that protein and refers to at least 3-5 amino acids, at least 8-10 amino acids, at least 11-15 amino acids, at least 17-24 amino acids, at least 25-30 amino acids, and at least 30-45 amino acids. In the case of oligonucleotides, a portion is defined by a contiguous portion of the nucleic acid sequence of that oligonucleotide and refers to at least 9-15 nucleotides, at least 18-30 nucleotides, at least 33-45 nucleotides, at least 48-72 nucleotides, at least 75-90 nucleotides, and at least 90-130 nucleotides. In some embodiments, portions of biomolecules have a biological activity. In the context of the present invention, FGF, e.g., an FGF21 polypeptide fragments do not comprise the entire FGF21 polypeptide sequence set forth in SEQ ID NO:2.

As used herein, the term “antibody” refers to monoclonal and polyclonal antibodies, single chain antibodies, chimeric antibodies, bifunctional/bispecific antibodies, humanized antibodies, human antibodies, and complementary determining region (CDR)-grafted antibodies, that are specific for the target protein or fragments thereof The term “antibody” further includes in vivo therapeutic antibody gene transfer. Antibody fragments, including Fab, Fab′, F(ab′)2, scFv, and Fv are also provided by the invention.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567), The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences.

“Antibody fragments” comprise a portion of an intact antibody, preferably comprising the antigen-binding or variable region thereof Examples of antibody fragments include Fab, Fab′, F(ab′)2, and Fv fragments; diabodies; linear antibodies (Zapata et al., Protein Eng. 8(10): 1057-1062 [1995]); single-chain antibody molecules; and multispecific antibodies formed from antibody fragment(s).

An “intact” antibody is one that comprises an antigen-binding variable region as well as a light chain constant domain (C_{L}) and heavy chain constant domains, C_{H1}, C_{H2} and C_{H3}. The constant domains may be native sequence constant domains (e.g. human native sequence constant domains) or amino acid sequence variants thereof. In some embodiments, the intact antibody has one or more effector functions.

Antibody “effector functions” refer to those biological activities attributable to the Fc region (a native sequence Fc region or amino acid sequence variant Fe region) of an antibody. Examples of antibody effector functions include Clq binding; complement dependent cytotoxicity; Fe receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” or “ADCC” refers to a form of cytotoxicity in which secreted 1 g bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hernatopoietic cells is summarized in Table 3 on page 464 of Ravetch and Kinet, Annu. Rev. Immunol. 9;457-92 (1991), To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, in a animal model such as that disclosed in Clynes et al. (USA) 95:652-656 (1998),

“Human effector cells” are leukocytes that express one or more FeRs and perform effector functions. In some embodiments, the cells express at least FcγRIII and perform ADCC effector function. Examples of human leukocytes that mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” or “FcR” are used to describe a receptor that binds to the Fc region of an antibody. The preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daron, Annu. Rev. Immunol. 15:203-234 (1997)). FcRs are reviewed in Ravetch and Kinet, Annu, Rev. Immunol. 9:457-92 (1991); Capel et Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)),

“Complement dependent cytotoxicity” or “CDC” refers to the ability of a molecule to lyse a target in the presence of complement. The complement activation pathway is initiated by the binding of the first component of the complement system (Clq) to a molecule (e.g. an antibody) complexed with a cogitate antigen. To assess complement activation, a CDC assay, e.g. as described in Gazzano-Santoro et al., J. Immunol. Methods 202:163 (1996), may be performed.

As used herein, the term “epitope” refers to an antigenic determinant of a polypeptide. In some embodiments an epitope may comprise 3 or more amino acids in a spatial conformation which is unique to the epitope. In some embodiments epitopes are linear or conformational epitopes. Generally an epitope consists of at least 4, at least 6, at least 8, at least 10, and at least 12 such amino acids, and more usually, consists of at least 8-10 such amino acids. Methods of determining the spatial conformation of amino acids are known in the art, and include, for example, x-ray crystallography and 2-dimensional nuclear magnetic resonance.

As used herein, the term “epitope bearing fragment” refers to a fragment of a polypeptide that includes one or more epitopes. In some embodiments, the epitope bearing fragment is not the full-length polypeptide.

The phrase “complementarily determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected front any of the five isotypes: alpha, delta, epsilon, gamma or mu. One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell tvceptors. See, e.g., U.S. Pat. Nos. 5,530,101 and 5,585,089 which are incorporated herein by reference.

The term “antagonist” is used in the broadest sense, and includes any molecule that partially or fully blocks, inhibits, or neutralizes a biological activity of a tumor cell antigen disclosed herein. In a similar manner, the term “agonist” is used in the broadest sense and includes any molecule that mimics a biological activity of a tumor cell antigen disclosed herein. Suitable agonist or antagonist molecules specifically include agonist or antagonist antibodies or antibody fragments, fragments or amino acid sequence variants of tumor cell antigens, peptides, antisense oligonucleotides, small organic molecules, etc. Methods for identifying agonists or antagonists of a tumor cell antigen may comprise contacting a tumor cell expressing the antigen of interest with a candidate agonist or antagonist molecule and measuring a detectable change in one or more biological activities normally associated with the tumor cell antigen. The antagonist may also be a peptide generated by rational design or by phase display (see, e.g., WO98/35036 published 13 Aug. 1998). In some embodiments, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide

As used herein, the term “oligonucleotide” refers to a series of linked nucleotide residues. Oligonucleotides include without hmitation, antisense and siRNA oligonucleotides. Oligonucleotides comprise portions of a DNA sequence and have at least about 10 nucleotides and as many as about 500 nucleotides. In some embodiments oligonucleotides comprise from about 10 nucleotides to about 50 nucleotides, from about 15 nucleotides to about 30 nucleotides, and from about 20 nucleotides to about 25 nucleotides. Oligonucleotides may be chemically synthesized and can also be used as probes. In some embodiments oligonucleotides are single stranded. In some embodiments oligonucleotides comprise at least one portion which is double stranded. In some embodiments the oligonucleotides are antisense oligonucleotides (ASO), in sonic embodiments the oligonucleotides are RNA interference. oligonucleotides (RNAi oligonucleotides).

As used herein, the term “therapeutically effective amount” is meant to refer to an amount of a medicament which produces a medicinal effect observed as reduction or reverse in one or more clinical endpoints, growth and ./or survival of cancer cell, or metastasis of cancer cells in an individual when a therapeutically effective amount of the medicament is administered to the individual. Therapeutically effective amounts are typically determined by the effect they have compared to the effect observed when a composition which includes no active ingredient is administered to a similarly situated individual. The precise effective amount for a subject will depend upon the subject's size and health, the nature and extent of the condition, and the therapeutics or combination of therapeutics selected for administration. However, the effective amount for a given situation is determined by routine experimentation and is within the judgment of the clinician.

As used herein, the terms “in combination with” or “in conjunction with” refer to administration of the FGF, e.g., the FGF21 agonists of the invention with other therapeutic regimens.

As used herein, the term “susceptible” refers to patients for whom FGF, e.g., an FGF21 therapy is an acceptable method of treatment, i.e., patients who are likely to respond positively. Cancer patients susceptible to FGF, e.g, an FGF21 agonist therapy express low levels of AMPK relative to those patients not susceptible to FGF, e.g., an FGF21 agonist therapy. Cancer patients who are not good candidates for FGF, e.g., an FGF21 agonist therapy include cancer patients with tumor samples that have normal to elevated levels of AMPK in or on their cancer cells.

As used herein the term “detecting” means to establish, discover, or ascertain evidence of an activity (for example, gene expression) or biomolecule (for example, a polypeptide).

A “native sequence” polypeptide is one that has the same amino acid sequence as a polypeptide derived from nature. Such native sequence polypeptides can be isolated from nature or can be produced hs recombinant or synthetic means. Thus, a native sequence polypeptide can have the amino acid sequence of naturally occurring human polypeptide, murine, polypeptide, or polypeptide from any other mammalian species.

The term “amino acid sequence variant” refers to polypeptides having amino acid sequences that differ to some extent from a native sequence polypeptide. Ordinarily, amino acid sequence variants will possess at least about 85%, at least about 90%, at least about 95%, at least about 98% or at least about 99% homology with at least one receptor binding domain of a native ligand or with at least one ligand binding domain of a native receptor. The amino acid sequence variants possess substitutions, deletions, and/or insertions at certain positions within the amino acid sequence of the native amino acid sequence.

As used herein, the phrase “homologous nucleotide sequence,” or “homologous amino acid sequence,” or variations thereof, refers to sequences characterized by a homology, at the nucleotide level or amino acid level, of at least a specified percentage and is used interchangeably with “sequence identity”. Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than humans, including, but not limited to, mammals. Homologous nucleotide sequences also include, but are not limited to, naturally occurring allelic variations and mutations of the nucleotide sequences set forth herein. Homologous amino acid sequences include those amino acid sequences which contain conservative amino acid substitutions and which polypeptides have the same binding and/or activity. In some embodiments, a nucleotide or amino acid sequence is homologous if it has at least 60%, 70%, 80%, 85%, 90%, 95%, 96%, 97%, 98%, or 99% identity. In some embodiments, a nucleotide or amino acid sequence is homologous if it has 1-10, 10-20, 20-30, 30-40, 40-50, or 50-60 nucleotide/amino acid substitutions, additions, or deletions. In some embodiments, the homologous amino acid sequences have no more than 5 or no more than 3 conservative amino acid substitutes.

Percent homology or identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, homology between the probe and target is between about 75% to about 85%. In some embodiments, nucleic acids have nucleotides that are at least about 85%, about 90%, about 92%, about 94%, about 95%, about 97%, about 98%, about 99% and about 100% homologous to SEQ ID NO:1, or a portion thereof.

Homology may also be at the polypeptide level. In some embodiments, polypeptides are about 85%, about 90%, about 92%, about 94%, about 95%, about 97%, about 98%, about 99% and about 100% homologous to SEQ ID NO:2, or a portion thereof. In some embodiments the polypeptides have up to 5, up to 10, up to 15, up to 20 or up to 30 amino acid insertions, deletions or substitutions.

As used herein, the term “probe” refers to nucleic acid sequences of variable length. In some embodiments probes comprise at least about 10 and as many as about 6,000 nucleotides. In some embodiments probes comprise at least 12, at least 14, at least 16, at least 18, at least 20, at least 25, at least 50 or at least 75 consecutive nucleotides. Probes are used in the detection of identical, similar, or complementary nucleic acid sequences. Longer length probes are usually obtained from natural or recombinant sources, are highly specific to the target sequence, and are much slower to hybridize to the target than are oligomers. Probes may be single- or double-stranded and are designed to have specificity in PCR, hybridization membrane-based, in situ hybridization (ISH), fluorescent in situ hybridization (FISH), or ELISA-like technologies.

As used herein, the term “mixing” refers to the process of combining one or more compounds, cells, molecules, and the like together in the same area. This may be performed, for example, in a test tube, petri dish, or any container that allows the one or more compounds, cells, or molecules, to be mixed.

As used herein the term “isolated” refers to a polynucicotide, a polypeptide, an antibody, or a host cell that is in an environment different from that in which the polynucleotide, the polypeptide, or the antibody naturally occurs. Methods of isolating cells are well known to those skilled in the art. A polynucleotide, a polypeptide, or an antibody which is isolated is generally substantially purified.

As used herein, the term “substantially purified” refers to a compound (e.g., either a polynucleotide or a polypeptide or an antibody) that is removed from its natural environment and is at least 60% free, at least 75% free, and at least 90% free from other components with which it is naturally associated.

As used herein, the term “binding” means the physical or chemical interaction between two or more biomolecules or compounds. Binding includes ionic, non-ionic, hydrogen bonds, Van der Waals, hydrophobic interactions, etc. Binding can be either direct or indirect; indirect being through or due to the effects of another biomolecule or compound. Direct binding refers to interactions that do not take place through or due to the effect of another molecule or compound but instead are without other substantial chemical intermediates.

As used herein, the term “contacting” means bringing together, either directly or indirectly, one molecule into physical proximity to a second molecule. The molecule can be in any number of buffers, salts, solutions, etc. “Contacting” includes, for example, placing a polynucleotide into a beaker, microtiter plate, cell culture flask, or a microarray, or the like, which contains a nucleic acid molecule. Contacting also includes, for example, placing an antibody into a beaker, inicrotiter plate, cell culture flask, or microarray, or the like, which contains a polypeptide. Contacting may take place in vivo, ex vivo, or in vitro.

As used herein, the phrase “stringent hybridization conditions” or “stringent conditions” refers to conditions under which a probe, primer, or oligonucleotide will hybridize to its target sequence, but to a minimal number of other sequences. Stringent conditions are sequence-dependent and will be different in different circumstances. Longer sequences will hybridize with specificity to their proper complements at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tₘ, 50% of the probes are hybridized to their complements at equilibrium. .rypically, stringent conditions will be those in which the salt concentration is less than about 1.0 M sodium ion, typically about 0.01 to 1.0 M sodium ion for other salts) at pH 7.0 to 8.3 and the temperature is at least about 30° C. for short probes, primers or oligonucleotides 10 to 50 nucleotides) and at least about 60° C. for longer probes, primers or oligonucleotides. Stringent conditions may also be achieved with the addition of destabilizing agents, such as formamide.

As used herein, the term “moderate stringency conditions” refers to conditions under which a probe, primer, or oligonucleotide will hybridize to its target sequence, but to a limited number of other sequences, Moderate conditions are sequence-dependent and will be different in different circumstances. Moderate conditions are well-known to the art skilled and are described in, inter alia, Maniatis et al. (Molecular Cloning: A Laboratory Manual, Cold Spring Harbor Laboratory; 2nd Edition (December 1989)).

The nucleic acid compositions described herein can be used, for example, to produce polypeptides, as probes for the detection of mRNA in biological samples (e.g., extracts of human cells) or cDNA produced from such samples, to generate additional copies of the polynucleotides, to generate ribozymes or oligonucleotides (single and double stranded), and as single stranded DNA probes or as triple-strand forming oligonucleotides. The probes described herein can be used to for example, determine the presence or absence of the polynucleotides provided herein in a sample. The polypeptides can be used to generate antibodies specific for a polypeptide associated with cancer, which antibodies are in turn useful in diagnostic methods, prognostic methods, and the like as discussed in more detail herein. Polypeptides are also useful as targets for therapeutic intervention, as discussed in more detail herein. Antibodies of the present: invention may also be used, for example, to purify, detect, and target the polypeptides of the present invention, including both in vitro and in vivo diagnostic and therapeutic methods. For example, the antibodies are useful in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed 1988). These and other uses are described in more detail below.

As used herein the term “imaging agent” refers to a composition linked to an antibody, small molecule, or probe of the invention that can be detected using techniques known to the art-skilled. As used herein, the term “evidence of gene expression” refers to any measurable indicia that a gene is expressed,

The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolind macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents pH buffering substances, and the like, can also be present in such vehicles.

“Cancer” as used herein, includes but is not limited to colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma and melanoma,

AMP-Activated Protein Kinase (AMPK)

AMPK belongs to the ser/thr protein kinase family. It consists of one catalytic (a) and two regulatory subunits (β and γ). The kinase activity of AMPK is activated by the stimuli that increase the cellular AMP/ATP ratio. AMPK is involved in metabolism and is a major regulator of energy homeostasis through the autophagic recycling of intracellular components.

AMPK is a metabolic sensor that is conserved in eukaryotes. Activation of AMPK acts to maintain cellular energy stores, switching on catabolic pathways that produce ATP, by enhancing oxidative metabolism and mitochondrial biogenesis, while turning off anabolic pathways that consume ATP. (Hardie, D G (2007) Nature Reviews Molecular Cell Biology 8:774-785) Recent evidence demonstrated the importance of AMPK in the therapeutic benefits of metformin, thiazolidinediones and exercise, which form the cornerstones of the clinical management of type 2 diabetes and associated metabolic disorders. (Shaw, R J, et al. (2005) Science 310:1642-1646)(Zhou, G C, et al. (2001) Journal of Clinical Investigation 108:1167-1174)

Recent reports have also indicated that AMPK may also be a beneficial target for cancer treatment. Cancer cells have characteristic metabolic changes from normal cells and being a key metabolic regulator. AMPK may regulate the switch. AMPK may act to inhibit tumorigenesis through regulation of cell growth, cell proliferation, autophagy, stress responses and cell polarity.

LKB1 mutations typically occur in non-small-cell lung cancers where ˜50% of the tumors harbor inactivating mutations, thus repressing its ability to activate AMPK. Activation of AMPK inhibits mTOR function and suppresses tumorigenesis-induced by PTEN loss “in vivo”

AMPK has also been shown to regulate cell polarity and mitosis, two energy-dependent processes. Tight control of mitosis by AMPK may prevent the occurrence of genetic instability and neoplastic development.

AMPK activation is also thought to prevent tumor growth arrest arrest via transcriptional induction and phosphorylation of p53. In melanoma, V600EBRAF abrogates LKB1/AMPK axis by activating ERK & RSK that phosphorylate and inhibit LKB1

Patients with type-2 diabetes taking metformin (AMPK activator) had a 23% reduced risk of developing cancer compared to those taking sulfonylurea. In a population-based cohort of 10,309 new users of metformin or sulfonylurea, the later group had a significant higher risk of cancer related mortality compared with the metformin group

Treatment of Cancers

For the treatment of diseases or disorders associated with underexpression of AMPK, such as a cancer, the present invention provides methods including other active ingredients in combination with the FGF, e.g., an FGF21 agonists of the present invention. In some embodiments, the methods further comprise administering one or more conventional cancer therapeutics to the patient. In some embodiments the methods of the present invention further comprise treating the patient with one or more of chemotherapy, radiation therapy, hormone ablation, or surgery.

The present invention also provides methods and compositions for the treatment, inhibition, and management of cancer or other hyperproliferative cell disorder or diseases that has become partially or completely refractory to current or standard cancer treatment, such as surgery, chemotherapy, radiation therapy, hormonal therapy, and biological therapy.

The invention also provides diagnostic and/or imaging methods using the FGF, e.g., an FGF21 agonists of the invention, to diagnose cancer and/or predict cancer progression. In some embodiments, the methods of the invention provide methods of imaging and localizing tumors and/or metastases and methods of diagnosis and prognosis. In some embodiments, the methods of the invention provide methods to evaluate the appropriateness of FGF, e.g., an FGF21-agonist related therapy.

Antibodies

In some embodiments the FGF, e.g., an FGF21 agonist is a monoclonal antibody. a polyclonal antibody, a chimeric antibody, a human antibody, a humanized antibody, a single-chain antibody, or a Fab fragment. The antibody may be labeled with, for example, an enzyme, radioisotope, or fluorophore. In some embodiments the antibody has a binding affinity less than about 1×10⁵Ka for a polypeptide other than FGF, e.g., an FGF21. In some embodiments, the FGF, e.g., an FGF21 modulator is a monoclonal antibody which binds to FGF, e.g., an FGF21 with an affinity of at least 1×10⁵Ka.

In some embodiments the antibody is a humanized antibody. Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed. which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger,. Nature Biotechnology 14, 826 (1996); Lonberg and Huszar, Intern. Rev. Immunol. 13 65-93 (1995); Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al, Science 239:1534-1536 (1988); Padlan. Molec. Immun. 28:489-498 (1991); Padlan, Molec. Immunol. 31(3): 169-217 (1994); and Kettleborough, C. A. et al., Protein Eng, 4(7);773-83 (1991) each of which is incorporated herein by reference.

Antibodies of the present invention may function through different mechanisms. In some embodiments antibodies trigger antibody dependent cellular cytotoxicity (ADCC), a lytic attack on antibody-targeted cells. In sonic embodiments, antibodies have multiple therapeutic, functions, including, fbr example, antigen-binding, induction of apoptosis, and complement-dependent cellular cytotoxicity (CDC).

In some embodiments, antibodies of the present invention may act as agonists or antagonists of the polypeptides of the present invention, For example, in some embodiments the present invention provides antibodies which disrupt the receptor ligand interactions with the polypeptides of the invention either partially or fully. In some embodiments antibodies of the present invention bind an epitope disclosed herein, or a portion thereof. In some embodiments, antibodies are provided that modulate ligand activity or receptor activity by at least 95%, at least 90%., at least 85%, at least 80%, at least 75%, at least 70%, at least 60%, or at least 50% compared to the activity in the absence of the antibody.

In some embodiments the antibodies may be specified as agonists, antagonists or inverse agonists for biological activities comprising the specific biological activities of the peptides of the invention disclosed herein.

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins, See, e.g., PCT publications WO 92/08495; WO 91/14438; WO 89/12624; U.S. Pat. No. 5,314,995; and EP 396,387.

In addition to chimeric and humanized antibodies, fully human antibodies can be derived from transgenic mice having human immunoglobulin genes (see, e.g., U.S. Pat. Nos. 6,075,181, 6,091.001, and 6,114,598, all of which are incorporated herein by reference), or from phage display libraries of human immunoglobulin genes (see, e.g. McCafferty et al., Nature, 348:552-554 (1990). Clackson et al., Nature, 352:624-628 (1991), and Marks et al., J. Mol. Biol., 222:581-597 (1991)). In some embodiments, antibodies may be produced and identified by scFV-phage display libraries. Antibody phage display technology is available from commercial sources such as from Xoma (Berkeley, Calif.).

Monoclonal antibodies can be prepared using the method of Kohler et al. (1975) Nature 256;495496, or a modification thereof: Typically, a mouse is immunized with a solution containing an antigen. Immunization can be performed by mixing or emulsifying the antigen-containing solution in saline, preferably in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial or limiting dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture bottles or hollow fiber reactors), or in vivo (as ascites in mice).

As an alternative to the use of hybridomas for expression, antibodies can be produced in a cell line such as a CHO or myeloma cell line, as disclosed in U.S. Pat. Nos. 5,545,403; 5,545,405; and 5,998,144; each incorporated herein by reference. Briefly the cell line is transfected with vectors capable of expressing a light chain and a heavy chain, respectively. By transfecting the two proteins on separate vectors, chimeric antibodies can be produced. Immunol. 147:8; Banchereau et al. (1991) Clin. Immunol. Spectrum 3:8; and Banchereau et al. (1991) Science 251:70; all of which are herein incorporated by reference.

Human antibodies can also be produced using techniques known in the art, including phage display libraries [Hoogenboom and Winter, J. Mol. Biol., 227:381 (1991); Marks et al., J. Mol, Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86 95 (1991)]. Humanized antibodies may be achieved by a variety of methods including, for example: (1) grafting the non-human complementarily determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779 783 (1992); Lonberg et al., Nature 368 856 859 (1994); Morrison, Nature 368, 812 13 (1994); Fishwild et al., Nature Biotechnology 14, 845 51 (1996); Neuberger, Nature Biotechnology 14, 826 (1996); Lonberg and Huszar, Intern. Rev. Immunol. 13 65 93 (1995); Jones et al., Nature 321;522-525 (1986); Morrison et al., Proc. Natl. Acad. Sci, 81:6851-6855 (1984); Morrison and Oi, Adv. Immunol. 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough. C. A. et al., Protein Eng. 4(7):773-83 (1991) each of which is incorporated herein by reference. Fully humanized antibodies can be identified in screening assays using commercial resources such as Morphosys (Martinsried/Planegg, Germany).

Humanized antibodies can also be produced using transgenic animals that are engineered to contain human immunoglobulin loci. For example, WO 98/24893 discloses transgenic animals having a human 1 g locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen. wherein the antibodies have primate constant and/or variable regions, and wherein the endogenous immunoglohulin-encoding loci are substituted or inactivated. WO 96/30498 discloses the use of the Cre/Lox system to modify the immunoglohulin locus in a mammal, such as to replace all or a portion of the constant or variable region to form a modified antibody molecule. WO 94/02602 discloses non-human mammalian hosts having inactivated endogenous 1 g loci and functional human 1 g loci. U.S. Pat. No. 5,939,598 discloses methods of making transgenic mice in which the mice lack endogenous heavy chains, and express an exogenous immunoglobulin locus comprising one or more xenogeneic constant regions. Antibodies of the present invention can also be produced using human engineering techniques as discussed in U.S. Pat. No. 5,756,886, which is incorporated herein by reference.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

Antibodies of the present invention may be administered to a subject via in viva therapeutic antibody gene transfer as discussed by Fang et at (2005), Nat. Biotechnol. 23, 584-590. For example recombinant vectors can be generated to deliver a multicistronic expression cassette comprising a peptide that mediates enzyme independent, cotranslational self cleavage of polypeptides placed between MAb heavy and light chain encoding sequences. Expression leads to stochiometric amounts of both MAb chains. A preferred example of the peptide that mediates enzyme independent, cotranslational self cleavage is the foot-and-mouth-disease derived 2A peptide.

Fragments of the antibodies are suitable for use in the methods of the invention so long as they retain the desired affinity of the full-length antibody. Thus, a fragment of an anti FGF, e.g, an FGF21 antibody will retain the ability to bind to FGF, e.g., an FGF21. Such fragments are characterized by properties similar to the corresponding ful length anti-FGF, e.g., an FGF21 antibody, that is, the fragments will specifically bind a human FGF, e.g., an FGF21 antigen expressed on the surface of a human cell.

In some embodiments the antibodies inhibit one or more of cancer cell growth, tumor formation, and cancer cell proliferation.

In some embodiments, the antibody is a monoclonal antibody which binds to one or more FGF, e.g., an FGF21 epitopes in a domain selected from the group consisting of the N-terminal signal peptide domain of FGF, e.g., an FGF21, or the receptor binding domain of FGF, e.g., an FGF21.

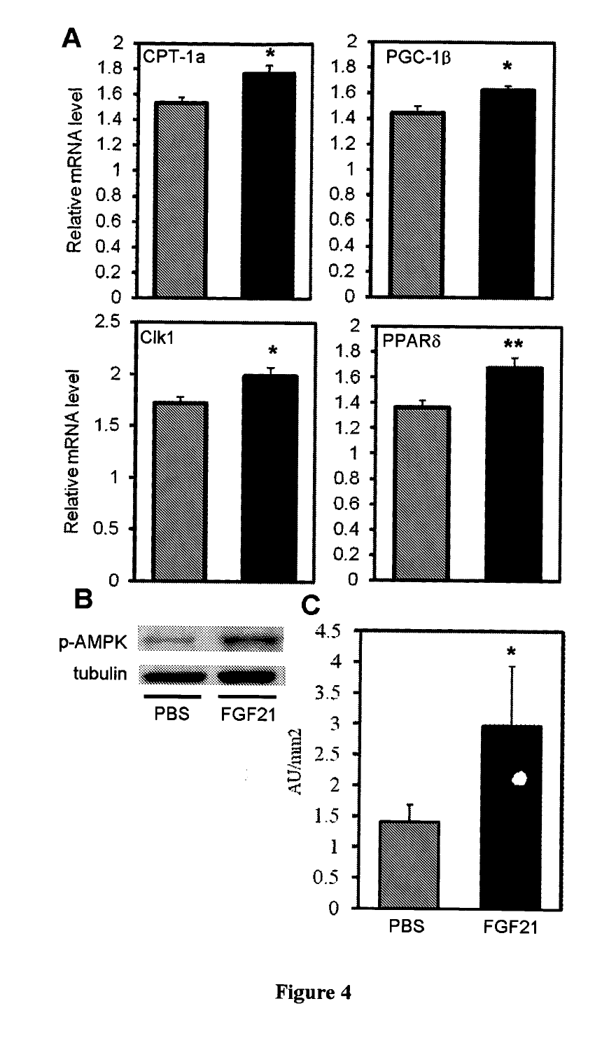

In some embodiments, the monoclonal antibody binds to an FGF, e.g., an FGF21 epitope in the C-terminal domain of FGF, e.g., an FGF21. In some embodiments, the monoclonal antibody binds to an FGF, e.g., an FGF21 epitope in region 2 or region 5 of FGF, e.g., an FGF21 as indicated in Example 4 (see also FIG. 4).

Suitable antibodies according to the present invention can recognize linear or conformational epitopes, or combinations thereof. In some embodiments the antibodies of the present invention bind to epitopes of antigenic regions of FGF, e.g., an FGF21 selected from the group consisting of SEQ ID NOs:3-203. In some embodiments the antibody is specific for an epitope having a sequence selected from the group consisting of SEQ ID NOs:117-203. In some embodiments the antibody is specific for an epitope having a sequence selected from the group consisting of SEQ ID NOS:186-203. It is to be understood that these peptides may not necessarily precisely map one epitope, but may also contain FGF, e.g., an FGF21 sequence that is not immunogenic.

Methods of predicting other potential epitopes to which an antibody of the invention can bind are well-known to those of skill in the art and include without limitation, Kyte-Doolittle Analysis (Kyte, J. and Doliftle, R. F., J. Mol. Biol. (1982) 157:105-132), Hopp and Woods Analysis (Hopp, T. P. and Woods, K. R., Proc. Natl. Acad. Sol. USA (1981) 78:3824-3828; Hopp, T. J. and Woods, K. R. Mol. Immunol. (1983) 20:483-489; Hopp, T. J., J. Immunol. Methods (1986) 88:1-18), Jameson-Wolf Analysis (Jameson, B. A. and Wolf H., Comput. Appl. Biosci. (1988) 4:181-186.), and Emini Analysis (Emini, E. A., Schlief, W. A., Colonno, R. J. and Wimmer, E., Virology (1985) 140:13-20.).

In some embodiments, potential epitopes are identified by determining theoretical extraeellular domains. Analysis algorithms such as TMpred (see K. Hofmann & W. Stoffel (1993) TMbase—A database of membrane spanning proteins segments Biol. Chem. Hoppe-Seyler 374,166) or TMHMM (Krogh et al., Predicting transmembrane protein topology with a hidden Markov model: Application to complete genomes. Journal of Molecular Biology, 305:567-580, 2001) can be used to make such predictions. Other algorithms, such as SignalP 3.0 (Bednsten et al., J Mol Biol. 340:783-95, 2004) can be used to predict the presence of signal peptides and to predict where those peptides would be cleaved from the full-length protein. The portions of the proteins on the outside of the cell can serve as targets for antibody interaction.

Antibodies are defined to be “specifically binding” if: 1) they exhibit a threshold level of binding activity, and/or 2) they do not significantly cross-react with known related polypeptide molecules. The binding affinity of an antibody can be readily determined by one of ordinary skill in the art, for example, by Scatchard analysis (Scatchard, Ann. NY Acad. Sci, 51 660-672, 1949). In some embodiments the antibodies of the present invention bind to then target epitopes or mimetic decoys at least 1.5-fold, 2-fold, 5-fold 10-fold, 100-fold, 10³-fold, 10⁴-fold, 10⁵-fold, 10⁶-fold or greater for the target cancer-associated polypeptide.

In some embodiments the antibodies bind with high affinity of 10⁻⁴M or less, 10⁻⁷M or less, 10⁻⁹M or less or with subnanomolar affinity (0.9, 0.8, 0.7, 0.6, 0.5, 0.4, 0.3, 0.2, 0.1 nM or even less). In some embodiments the binding affinity of the antibodies for FGF, e.g., all FGF21 is at least 1×10⁶ Ka. In some embodiments the binding affinity of the antibodies for FGF, e.g., an FGF21 is at least 5×10⁶ Ka, at least 1×10⁷ Ka, at least 2×10⁷ Ka, at least 1×10⁸ Ka, or greater. Antibodies of the present invention may also be described or specified in terms of their binding affinity to a polypeptide of the invention. In some embodiments binding affinities include those With a Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M, or less.

In some embodiments, the antibodies of the present invention bind FGF, e.g., an FGF21 polypeptide but not known related family members of FGF, e.g., an FGF21. Binding of an antibody to FGF, e.g., an FGF21 and related polypeptides an be assayed using standard Western blot analysis (Ausubel et al.). Examples of known related polypeptides include, without limitation, other members of the FGF protein family (e, FGF19x (WO 01/18209), and the like).

In some embodiments, the antibodies of the present invention bind to orthologs, homologs, paralogs or variants, or combinations and subcombinations thereof, of FGF, e.g., an FGF21. In some embodiments, the antibodies of the present invention bind to orthologs of FGF, e.g., an FGF21, In sonic embodiments, the antibodies of the present invention bind to homologs of FGF, e.g., an FGF21. In some embodiments, the antibodies of the present invention bind to paralogs of FGF, e.g., an FGF21. In some embodiments, the antibodies of the present invention hind to variants of FGF, e.g., an FGF21. In some embodiments, the antibodies of the present invention do not bind to orthologs, homologs, paralogs or variants, or combinations and subcombinations thereof, of FGF, e.g., an FGF21. In some embodiments, the antibodies of the present invention do not specifically bind to FGF19x.

In some embodiments, antibodies may be screened against known related polypeptides to isolate an antibody population that specifically binds to FGF, e.g., an FGF21 polypeptides. For example, antibodies specific to human FGF, e.g., an FGF21 polypeptides will flow through a column comprising other FGF proteins (FGF19x, and the like) adhered to insoluble matrix under appropriate buffer conditions. Such screening allows isolation of polyclonal and monoclonal antibodies non-crossmactive to closely related polypeptides (Antibodies A Laboratory Manual Harlow and Lane (eds.), Cold Spring Harbor Laboratory Press, 1988; Current Protocols in Immunology, Cooligan et al. (eds.), National Institutes of Health, John Wiley and Sons, Inc., 1995). Screening and isolation of specific antibodies is well known in the art (see, Fundamental Immunolog), Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984). Representative examples of such assays include: concurrent immunoelectrophoresis, radioimmunoassay (RIA), radioimmunoprecipitation, enzyme-linked immunosorbent assay (ELISA), dot blot or Western blot assay, inhibition or competition assay, and sandwich assay.

In some embodiments, the antibodies of the present invention do not specifically bind to epitopes consisting of a sequence selected from the group consisting of SEQ ID NOs:3-111, SEQ ID NOs:133-138, or SEQ ID NOs:160-164 (Table 2). In some embodiments, the antibodies of the present invention do not specifically bind to epitopes consisting of residues 1-49 of SEQ ID NO:2. in some embodiments, the antibodies do not bind FGF19x.

The invention also provides antibodies that are SMIPs or binding domain immunoglobulin fusion proteins specific for target protein. These constructs are single-chain polypeptides comprising antigen binding domains fused to immunoglobulin domains necessary to carry out antibody effector functions. See e.g., WO03/041600, U.S. Patent Publication 20030133939 and U.S. Patent Publication 20030118592.

In some embodiments the antibodies of the present invention are neutralizing antibodies. A neutralizing antibody binds an infectious agent, such as a virus or a bacterium, such as a virus or bacterium associated with cancer a JC polyoma virus, Epstein-Barr virus, or Helicobacter pylori). In some embodiments the neutralizing antibodies can effectively act as receptor antagonists, inhibiting either all or a subset of the biological activities of the ligand-mediated receptor activation. In some embodiments the antibodies may be specified as agonists, antagonists or inverse agonists for biological activities comprising the specific biological activities of the peptides of the invention disclosed herein.

The antibodies of the present invention can be screened for the ability to either be rapidly internalized upon binding to the tumor-cell antigen in question, or for the ability to remain on the cell surface following binding. In some embodiments, for example in the construction of some types of immunocojugates, the ability of an antibody to be internalized may be desired if internalization is required to release the toxin moiety. Alternatively, if the antibody is being used to promote ADCC or CDC, it may be more desirable for the antibody to remain on the cell surface, A screening method can be used to differentiate these type behaviors. For example, a tumor cell antigen bearing cell may be used where the cells are incubated with human IgG1 (control antibody) or one of the antibodies of the invention at a concentration of approximately 1 μg/mL on ice (with 0.1% sodium azide to block internalization) or 37° C. (without sodium azide) for 3 hours. The cells are then washed with cold staining buffer (PBS+1% BSA+0.1% sodium azide), and are stained with goat anti-human IgG-FITC for 30 minutes on ice. Geometric mean fluorescent intensity (ME1) is recorded by FACS Calibur. If no difference in MF1 is observed between cells incubated with the antibody of the invention on ice in the presence of sodium azide and cells observed at 37° C. in the absence of sodium azide, the antibody will be suspected to be one that remains bound to the cell surface, rather than being internalized. If however, a decrease in surface stainable antibody is found Avhen the cells are incubated at 37° C. in the absence of sodium azide, the antibody will be suspected to be one which is capable of internalization.

Antibody Conjugates

In some einbodiments, the antibodies of the invention are conjugated. In some embodiments, the conjugated antibodies are useful for cancer therapeutics, cancer diagnosis, r imaging of cancerous cells.

For diagnostic applications, the antibody typically will be labeled with a detectable moiety. Numerous labels are available which can be generally grouped into the following categories:

(a) Radionuclides such as those discussed infra. The antibody can be labeled, for example, with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Fluorescent labels such as rare earth chelates (europium chelates) or fluorescein and its derivatives, rhodamine and its derivatives, dansyl, Lissmine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available and U.S. Pat. No. 4,275,149 presides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate, Techniques for quantifying a change in fluorescence am described above. The chemiluminescent substrate becomes electronically excited by a chemical reaction and may then emit light which can be measured (using a chemiluminometer, for e,xample) or donates energy to a fluorescent acceptor. Examples of enzymatic labels include luciferases (e.g., firefly luciferase and bacterial luciferase; U.S. Pat. No 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, micmperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym, (ed J. Langone & H. Van Vunakis), Academic press, New York, 73:147-166 (1981).

The antibodies may also be used for in vivo diagnostic assays. In some embodiments, the antibody is labeled with a radionuclide so that the tumor can be localized using immunoscintiography. As a matter of convenience, the antibodies of the present invention can be provided in a kit, i.e., a packaged combination of reagents in predetermined amounts with instructions for performing the diagnostic assay. Where the antibody is labeled with an enzyme, the kit may include substrates and cofactors required by the enzyme (e.g., a substrate precursor which provides the detectable chromophore or fluorophore). In addition, other additives may be included such as stabilizers, butTers (e.g., a block buffer or lysis buffer) and the like. The relative amounts of the carious reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In some embodiments, antibodies are conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52: 127-131 (1992)) to generate a maytansinoid-antibody immunoconjugate. In some embodiments, the conjugate may be the highly potent maytansine derivative DM1 (N2′-deacetyl-N2′-(3-mercapto-1-oxopropyl)-maytansine) (see for example WO02/098883 published Dec. 12, 2002) which has an IC50 of approximately 10-11 M (review, see Payne (2003) Cancer Cell 3:207-212) or DM4 (N2′-deacetyl-N2′(4-methyl-4-mercapto-1-oxopentyI)-maytansine) (see for example WO2004/103272 published Dec. 2, 2004).

In some embodiments the antibody conjugate comprises an anti-tumor cell antigen antibody conjugated to one or more calicheamicin molecules. The calicheamicin family of antibiotics is capable of producing double-stranded DNA breaks at sub-picomolar concentrations. Structural analogues of calicheamicin which may be used include, but are not limited to, gamma11, alpha21, alpha31, N-acetyl-gamma11, PSAG and thetaII (Hinman et al. Cancer Research 53: 3336-3342 (1993) and Lode et al. Cancer Research 58: 2925-2928 (1998)). See, also, U.S. Pat. Nos. 5,714,586; 5,712,374; 5,264,586; and 5,773,001, each of which is expressly incorporated herein by reference.

In some embodiments the antibody is conjugated to a prodrug capable of being released in its active form by enzymes overproduced in many cancers, For example, antibody conjugates can be made with a prodrug form of doxorubicin wherein the active component is released from the conjugate by plasmin. Plasmin is known to be over produced in many cancerous tissues (see Decy et al, (2004) FASEB Journal 18(3): 565-567).

In some embodiments the antibodies are conjugated to enzymatically, active toxins and fragments thereof. In some embodiments the toxins include, without limitation, diphtheria A chain, nonbinding active fragments of diphtheria toxin, exotoxin A chain (from Pseudomonas aeruginosa),Pseudomonas endotoxin, ricin A chain, abrin A chain, modeccin A chain, alpha-sarcin, Aleurites fordii proteins, dianthin proteins, Phytolaca americana proteins (PAPI, PAPII, and PAP-S), Ribonuclease (Rnase), Deoxyribonuclease (Dnase), pokeweed antiviral protein, momordica charantia inhibitor, curcin, crotin, sapaonaria officinalis inhibitor, gelonin, mitogellin, restrictocin, phenomycin, neomycin and the tricothecenes. See, for example, WO 93/21232 published Oct. 28, 1993. In some embodiments the toxins have low intrinsic immunogenicity and a mechanism of action (e.g. a cytotoxie mechanism versus a cytostatic mechanism) that reduces the opportunity for the cancerous cells to become resistant to the toxin.

In some embodiments conjugates are made between the antibodies of the invention and immunomodulators. For example, in some embodiments immunostimulatory oligonucleotides can be used. These molecules are potent immunogens that can elicit antigen-specific antibody responses (see Datta et al, (2003) Ann N.Y. Acad. Sci 1002: 105-111). Additional immunomodulatory compounds can include stem cell growth factor such as “S1 factor”, lymphotoxins such as tumor necrosis factor (TNF), hematopoietic factor such as an interleukin colony stimulating factor (CSF) such as granulocyte-colony stimulating factor (G-CSF) or granulocyte macrophage-stimulating factor (GM-CSF), interferon (IFN) such as interferon alpha, beta or gamma, erythropoietin, and thrombopoietin,

In some embodiments radioconjugated antibodies are provided. In some embodiments such antibodies can be made using ³²P, ³³P, ⁴⁷Sc, ⁵⁹Fe, ⁶⁴ Cu, ⁶⁷ Cu, ⁷⁵Se, ⁷⁷AS, ⁸⁹Sr, ⁹⁰Y, ⁹⁹Mo, ¹⁰⁵Rh, ¹⁰⁹Pd, ¹²⁵I, ¹³¹I, ¹⁴²Pr, ¹⁴³Pr, ¹⁴⁹Pm, ¹⁵³Sm, ¹⁶¹Th, ¹⁶⁶Ho, ¹⁶⁹Er, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹Pb, ²¹²Pb, ²¹³Bi, ⁵⁸Co, ⁶⁷Ga, ^{80m}Br, ^{99m}Tc, ^{103m}Rh, ¹⁰⁹Pt, ¹⁶¹Ho, ^{189m}Os, ¹⁹²Ir, ¹⁵²Dy, ²¹¹At, ²¹²Bi, ²²³Ra, ²¹⁹Rn, ²¹⁵Po, ²¹¹Bi, ²²⁵Ac, ²²¹Fr, ²¹⁷At, ²¹³Bi, ²⁵⁵Fm and combinations and subcombinations thereof. In some embodiments, boron, gadolinium or uranium atoms are conjugated to the antibodies, In some embodiments the boron atom is ¹⁰B, the gadolinium atom is ¹⁵⁷Gd and the uranium atom is ²³⁵U.

In some embodiments the radionuclide conjugate has a radionuclide with an energy between 20 and 10,000 keV. The radionuclide can be an Auger emitter, with an energy of less than 1000 keV, a P emitter with an energy between 20 and 5000 keV, or an alpha or ‘a’ emitter with an energy between 2000 and 10,000 keV.

In some embodiments diagnostic radioconjugates are provided which comprise a radionuclide that is a gamma-, beta-, or positron-emitting isotope. In some embodiments the radionuclide has an energy between 20 and 10,000 keV. In some embodiments the radionuclide is selected from the group of ¹⁸F, ⁵¹Mn, ^{52m}Mn, ⁵²Fe, ⁵⁵Co, ⁶²Cu, ⁶⁴Cu, ⁶⁸Ga, ⁷²As, ⁷⁵Br, ⁷⁶Br, ^{82m}Rb, ⁸³Sr, ⁸⁹Zr, ^{94m}Tc, ⁵¹Cr, ⁵⁷Co, ⁵⁸Co, ⁵⁹Fe, ⁶⁷Ga, ⁷⁵Se, ⁹⁷Ru, ^{99m}Tc, ^{114m}In, ¹²³I, ¹²⁵I, ¹³Li and ¹⁹⁷Hg.

In some embodiments the antibodies of the invention are conjugated to diagnostic agents that are photoactive or contrast agents. Photoactive compounds can comprise compounds such as chromagens or dyes. Contrast agents may be, for example a paramagnetic ion, wherein the ion comprises a metal selected from the group of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III). The contrast agent may also be a radio-opaque compound used in X-ray techniques or computed tomography, such as an iodine, iridium, barium, gallium and thallium compound. Radio-opaque compounds may be selected from the group of barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, Metrizamide, metrizoate, propyliodone, and thallous chloride. In some embodiments, the diagnostic immunoconjugates may contain ultrasound-enhancing agents such as a gas filled liposome that is conjugated to an antibody of the invention. Diagnostic immunoconjugates may be used for a variety of procedures including, but not limited to, intraoperative, endoscopic or intravascular methods of tumor or cancer diagnosis and detection,

In some embodiments antibody conjugates are made using a variety of bifunctional protein coupling agents such as N-succinimidyl-3-(2-pyridyldithiol) propionate (SPDP), succinimidyl-4-(N-maleimidomethyl) cyclohexane.-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzayl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987), Carbon-14-labeled 1-isothiocyanatobenzyl-3-methyldiethyllene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell, For example, an acid-labile linker, peptidase-sensitive linker, dimethyl linker or disulfide-containing linker (Chari et al. Cancer Research 52: 127-131 (1992)) may be used. Agents may be additionally linked to the antibodies of the invention through a carbohydrate moiety.

In some embodiments fusion proteins comprising the antibodies of the invention and cytotoxic agents may be made, e.g. by recombinant techniques or peptide synthesis. In some embodiments such immunoconjugates comprising the anti-tumor antigen antibody conjugated with a cytotoxic agent are administered to the patient. In some embodiments the immunoconjugate and/or tumor cell antigen protein to which it is bound is/are internalized by the cell, resulting in increased therapeutic efficacy of the immunoconjugate in killing the cancer cell to which it binds. In some embodiments, the cytotoxic agent targets or interferes with nucleic acid in the cancer cell. Examples of such cylotoxic agents include maytansinoids, calicheamicins, ribonucleases and DNA endonucleases.

In some embodiments the antibodies are conjugated to a “receptor” (such as streptavidin) for utilization in tumor pretargeting Wherein the antibody-receptor conjugate is administered to the patient, followed by rmoval of unbound conjugate from the circulation using a clearing agent and then administration of a “ligand” (e.g. avidin) which is conjugated to a cytotoxic agent (e.g. a radionucleotide).

In some embodiments the antibodies are conjugated to a cytotoxic molecule which is released inside a target cell lysozome. For example, the drug monomethyl auristatin E (MMAE) can be conjugated via a valine-citrulline linkage which will be cleaved by the proteolytic lysozomal enzyme cathepsin B following internalization of the antibody conjugate (see for example WO03/026577 published Apr. 3, 2003). In some embodiments, the MMAE can be attached to the antibody using an acid-labile linker containing a hydrazone functionality as the cleavable moiety (see for example WO02/088172 published Nov. 11, 2002).

Mimetics

In some embodiments, the FGF, e.g., an FGF21 agonist is a mimetic. As used herein, the term “mimetic” is used to refer to compounds which mimic the activity of a peptide. Mimetics are non-peptides but may comprise amino acids linked by non-peptide bonds, U.S. Pat. No. 5,637,677, issued on Jun. 10, 1997, and parent applications thereof all of which are incorporated herein by reference, contain detailed guidance on the production of mimetics. Briefly, the three-dimensional structure of the peptides which specifically interacts with the three dimensional structure of the FGF, e.g., an FGF21 is duplicated by a molecule that is not a peptide. In some embodiments the FGF, e.g., an FGF21 mimetic is a mimetic of FGF, e.g., an FGF21 or a mimetic of a ligand of FGF, e.g., an FGF21.

Fusion Proteins and FGF-Derived Peptidic Compounds

In another embodiment, the FGF, e.g., FGF21 agonist used in the methods of the present invention is a fusion protein or peptidic compound derived from the FGF, e.g., FGF21 amino acid sequence. Such fusion proteins and peptidic compounds can be made using standard techniques known in the art. For example, peptidic compounds can be made by chemical synthesis using standard peptide synthesis techniques and then introduced into cells by a variety of means known in the art for introducing peptides into cells (e.g., liposome and the like).

The in vivo half-life of the fusion protein or peptidic compounds of the invention can be improved by making peptide modifications, such as the addition of N-linked glycosylation sites into FGF, FGF21, or conjugating FGF, e.g., FGF21 to poly(ethylene glycol) (PEG; pegylation), e.g., via lysine-monopegylation. Such techniques have proven to be beneficial in prolonging the half-life of therapeutic protein drugs. It is expected that pegylation of the FGF, e.g., FGF21 agonists of the invention may result in similar pharmaceutical advantages.

In addition, pegylation can be achieved in any part of a polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No, 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The FGF, e.g., FGF21 agonist polypeptides containing these novel amino acids can then be pegylated at these chosen sites in the protein.

Methods of Treating/Preventing Cancer

The present invention provides methods for treating and/or preventing cancer or symptoms of cancer in a subject comprising administering to the subject a therapeutically effective amount of one or more FGF, e.g., an FGF21 agonists of the present invention. In some embodiments the cancer is a cancer associated with underexpmssion of AMPK. In some embodiments, the cancer is colon cancer, liver cancer, testicular cancer, thymus cancer, breast cancer, skin cancer, esophageal cancer, pancreatic cancer, prostatic cancer, uterine cancer, cervical cancer, lung cancer, bladder cancer, ovarian cancer, multiple myeloma or melanoma. In some embodiments, the cancer is in a non-hormonally regulated tissue. In some embodiments the subject has been diagnosed as having a cancer or as being predisposed to cancer.

Symptoms of cancer are well-known to those of skill in the art and include, without limitation, weight loss, anemia, abdominal pain, intestinal obstruction, blood in the stool, diarrhea, constipation, other changes in bowel habits, colon metastases, death, weakness, excessive fatigue, difficulty eating, loss of appetite, chronic cough, worsening breathlessness, coughing up blood, blood in the urine, nausea, vomiting, liver metastases, lung metastases, bone metastases, abdominal fullness, bloating, fluid in peritoneal cavity, vaginal bleeding, abdominal distension, perforation of colon, acute peritonitis (infection, fever, pain), pain, vomiting blood, heavy sweating, fever, high blood pressure, jaundice, dizziness, chills, muscle spasms, lung metastases, bladder metastases, liver metastases, bone metastases, kidney metastases, and pancreas metastases, difficulty swallowing, and the like.

A therapeutically effective amount of the agonizing compound can be determined empirically, according to procedures well known to medicinal chemists, and will depend, inter alia, on the age of the patient, severity of the condition, and on the ultimate pharmaceutical formulation desired. Administration of the agonists of the present invention can be carried out, for example, by inhalation or suppository or to mucosal tissue such as by lavage to vaginal, rectal, Urethral, buccal and sublingual tissue, orally, topically intranasally, intraperitoneally, parenterally, intravenously, intralymphatically, intratumorly, intramuscularly, interstitially, intra-arterially, subcutaneously, intraoccularly, intrasynovial, transepithelial, and transdermally. In some embodiments, the agonists are administered by lavage, orally or inter-arterially, Other suitable methods of introduction can also include rechargeable or biodegradable devices and slow or sustained release polymeric devices. As discussed above, the therapeutic compositions of this invention can also be administered as part of a combinatorial therapy with other known anti-cancer agents or other known anti-bone disease treatment regimen.

In further aspects, the present invention provides methods of modulating one or more activities in cells that express AMPK comprising contacting the cells with an amount of an FGF, e.g., an FGF21 agonist effective to modulate the one or more activities.

In some aspects, the present invention provides methods for detecting a tumor in a patient comprising administering to the patient a composition comprising an FGF, e.g., an FGF21 agonist linked to an imaging agent and detecting the localization of the imaging agent in the patient.

In some aspects, the present invention provides methods of identifying a cancer inhibitor, wherein the cancer is characterized by differential expression of AMPK compared to a control. The methods comprise contacting a cell expressing AMPK with a candidate FGF, e.g., an FGF21 agonist and determining whether an AMPK-related activity is modulated. Modulation of the AMPK-related activity is indicative of a cancer inhibitor.

In some aspects, the present invention provides methods for determining the susceptibility of a patient to an FGF, e.g., an FGF21 agonist comprising detecting evidence of underexpression of AMPK in a cancer sample of said patient compared to a control sample. Evidence of underexpression of AMPK is indicative of the patient's susceptibility to the FGF, e.g., an FGF21 agonist.

In some aspects, the present invention provides methods of delivering a cytotoxic agent or a diagnostic agent to one or more elk that express AMPK. The methods comprise (a) providing the cytotoxic agent or the diagnostic agent conjugated to an anti-FGF, e.g.. an FGF21 agonist; and (b) exposing the cell to the antibody-agent or fragment-agent conjugate.

In further aspects, the present invention provides methods for determining the effectiveness of a candidate FGF, e.g., an FGF21 agonist. The methods comprise contacting AMPK-expressing cells with the candidate FGF, e.g., an FGF21 agonist and detemiining whether a downstream marker of AMPK is modulated. Modulation of the downstream marker indicates that the candidate FGF, e.g., an FGF21 agonist is an effective anti-cancer medication,

In further aspects, the present invention provides methods of determining whether a cancer is susceptible to FGF, e.g., an FGF21-related treatment comprising comparing AMPK expression in cancer and control cells. Downregulation of AMPK expression in the cancer cells as compared to the control cells indicates that the cancer is susceptible to an FGF, e.g., an FGF21-related treatment.

In further aspects present meiflion provides methods of determining whether a cancer is susceptible to an FGF, e.g., an FGF21-related treatment comprising contacting a cancer sample with an FGF, e.g, an FGF21 agonist, and measuring an AMPK downstream marker in the cancer sample, Modulation of the downstream marker in the presence of the agonist as compared to the downstream marker in the absence of the agonist indicates that the cancer is susceptible to an FGF, e.g., an FGF21- related treatment.

In some aspects, the present invention provides methods of treating a cancer patient comprising determining whether a cancer is susceptible to an FGF, e.g., an FGF21-related treatment and administering to the patient an FGF, e.g., an FGF21 agonist if the patient has an cancer type which is susceptible to an FGF, e.g., an FGF21-related treatment, or administering to the patient a conventional cancer therapeutic if the patient does not have a cancer type which is susceptible to an FGF, e.g., an FGF21-related treatment.

### EXAMPLES

### Example 1

### FGF21 Enhances Mitochondrial Function Through an AMPK and PGC-1αDependent Pathway in Adipocytes

Research Design and Methods

Animals and treatments. Male ob/ob mice were obtained from Jackson Laboratories (Bar Harbor, Me.) at 8 weeks of age. Mice were randomly assigned to treatment or vehicle groups, and the randomization was stratified by body weight and fed blood glucose levels. Mice were treated with vehicle or FGF21 via continuous subcutaneous infusion with osmotic pumps (Azlet, Cupertino, Calif). Food and body weights were recorded daily. Blood samples were taken from conscious, fed animals by tail snip, and glucose levels were determined using Precision G Blood Glucose Testing System (Abbott Laboratories, Santa Clara, Calif.). A glucose tolerance test was performed after overnight fasting and challenged with a glucose load (1 g/kg).

Cell culture, in vitro treatments, and transfection experiments. 3T3-L1 cells were obtained from American Type Culture Collection. 3T3-L1 fibroblasts were grown to confluence in growth medium (Dulbecco's modified Eagle's medium supplemented With 10% fetal calf serum, 100 units/ml penicillin, and 100 μg/ml streptomycin). Differentiation was induced by incubating the cells in growth medium supplemented with 3-isobutyl-1-methylxanthine, dexamethasone, and insulin (Sigma Aldrich, St ,Louis, Mo.) for 4 days. The cells were further incubated in growth medium for an additional 4 days to complete adipocyte conversion. At day 8, cells were incubated in growth medium plus FGF21 (4.0 μg/ml) or Vehicle (PBS) for 72 h. Media containing FGF21 or vehicle control were replaced every 24 hours. Human adipocytes were obtained from Cell Applications, Inc (San Diego, Calif.). Human pre-adipocytes were grown to confluence in Pre-adipocytes medium. Differentiation was induced by incubating cells in adipocyte differentiation medium for 10 days. For knockdown experiments, 3T3-L1 adipocyteS were infected with adenovirus expressing shRNA constructs against SIRT1 (ViraQuest. North Liberty, Iowa), PGC-1α (Welgen, Worcester, Mass.) via lipofectamine 2000 (Invitrogen, Carlsbad, Calif.). Dominant-negative AMPK α2 adenovirus were purchased from Eton Bioscience (San Diego, Calif.), and amplified by ViraQuest.

Expression and purification of recombinant human FGF21. The cDNA encoding residues 33-209 of human FGF21 (SwissProt Q9NSA1) with an N-terminal 6×His tag followed by a TEV cleavage site was cloned into the NcoI and Xhol sites of pET-15b (Novagen, Madison, Wis.). The resulting plasmid was transformed into the E. coli Rosetta (DE3) pLysS strain (Novagen) and expression of recombinant human FGF-21 (rhFGF-21) was induced with IPTG for 5 hr at 37° C. His tag was removed by treatment with TEV protease. The sample was then concentrated in a 5K MWCO Amicon unit and injected onto a Superdex 200 gel filtration column (GE Healthcare, Piscataway, N.J.) equilibrated in PBS. The majority of eluted rhFGF21 was monomer and >99% pure based on Coomassie stained SDS-PAGE, The protein was stored m PBS. Protein concentration was determined using the Bradford protein assay according to the manufacturer's protocol (Bio-Rad, Hercules, Calif.).

RNA isolation, reverse transcription, and real-time quantitative PCR. RNA was isolated from cells and tissues using TRIzol reagent (Invitrogen, Carlsbad, Calif.), and purified with Qiagen RNeasy Mini kit (Qiagen, Valencia, Calif.). Isolated RNA was reverse transcribed into cDNA by following the protocol from SuperScript III First-Strand Synthesis System (Invitrogen). Real-time quantitative PCR reactions were performed in triplicate on an ABI Prism 7900HT (Applied Biosystems, Foster City, Calif.), and normalized to TATA box binding protein (Tbp). Assay-on-Demand Gene Expression primers and probes were obtained from Applied Biosystems.

Oxygen consumption and citrate syntbase activity assays. Citrate synthase activity was measured according to instructions provided with the citrate synthase kit obtained from Sigma-Aldrich. Briefly, cells were lysed in CellLytic M Cell lysis reagent, and protein concentration was determined using the BCA protein assay kit from Pierce (Rockford, Ill.). A total of 2 μg of protein was used for the assay. For oxygen consumption measurement, the XF24 Extracellular Flux Analyzer (Seahorse Biosciences, North Billerica, Mass.) was employed. Measurements were taken using 24 optical fluorescent biosensors embedded in a sterile disposable cartridge that is placed into the Seahorse 24-well tissue culture microplate. The oxygen consumption rate (OCR) was calculated using the fixed delta (50 mmHg) technique for determining the slope following the manufacturer's instructions. In brief, after measuring basal OCR 3 times, 10 μg/ml of oligomycin was injected (to make its final concentration 1.0 μg/ml). OCR was measured 3 times, followed by 3 more OCR readings after 3 μM of FCCP injection (to make its final concentration 300 nM).

NAD/NADH and CytC assays. NAD and NADH levels were determined according to instructions provided with the NAD/NADH assay kit purchased from Abcam (Cambridge, Mass.). CytC protein levels were measured using a CytC elisa assay obtained from R&D Systems (Minneapolis, Minn.).

Western blotting. Cells were lysed in Cell Lysis Buffer (Cell Signaling Technology, Danvers, Mass.) supplemented with protease and phosphatase inhibitors (Sigma Aldrich). Lysates were sonicated for 1 min and centrifuged at 14000 g for 10 min at 4° C. Lysates were resolved by 4-20% Tris-glycine SDS-PAGE and transferred to nitrocellulose membranes. Total and phospho-AMPK antibodies were obtained from Cell Signaling Technology.

Statistical analyses. Data are presented as mean ±SEM. Statistical analyses were performed using Student's t-test. Significant differences of p<0.05 are identified with an asterisk (*).

Results

FGF21 increases AMPK activity. To gain further insight into the mechanism by which FGF21 regulates energy expenditure, we treated 3T3-L1 adipocytes with FGF21 (4.0 μg/ml) for 3 days, and analyzed the levels of activated AMPK by Western blot. FGF21 treatment robustly increased the levels of AMPK phosphorylation (p-AMPK) by 53% (p<0.05, n=3), while total AMPK protein levels remained unchanged. Treatment of human differentiated adipocytes with FGF21 (4.0 μg/ml for 3 days) induced a significant increase in levels of p-AMPK by 58% (p<0.05, n=3), while total AMPK protein levels were not altered. Inhibition of AMPK activity in human adipocytes with adenovirus over-expressing the dominant-negative form of the alpha2 subunit of AMPK (DN-AMPK) reduced the FGF21-stimulated increase in p-AMPK.

To explore if FGF21 increases AMPK activity in vivo, we administered recombinant human FGF21 protein via continuous infusion with osmotic (Azlet) pumps to ob/ob mice for 2 weeks. Consistent with previous reports, FGF21 administration led to a significant reduction in total body weight, which was associated with a small, but significant decrease in food intake at days 10 and 14. Body/tissue composition measurements revealed a significant reduction in total body fat mass and liver lipid content in FGF21-treated animals. We analyzed the levels of phosphorylated AMPK in white adipose tissues from vehicle-and FGF21-treated animals and found increased levels of p-AMPK (53%) (p<0.05, n=8) in FGF21-treated mice, indicating enhanced activation of AMPK. These data suggest that FGF21 regulates energy expenditure through activation of AMPK,

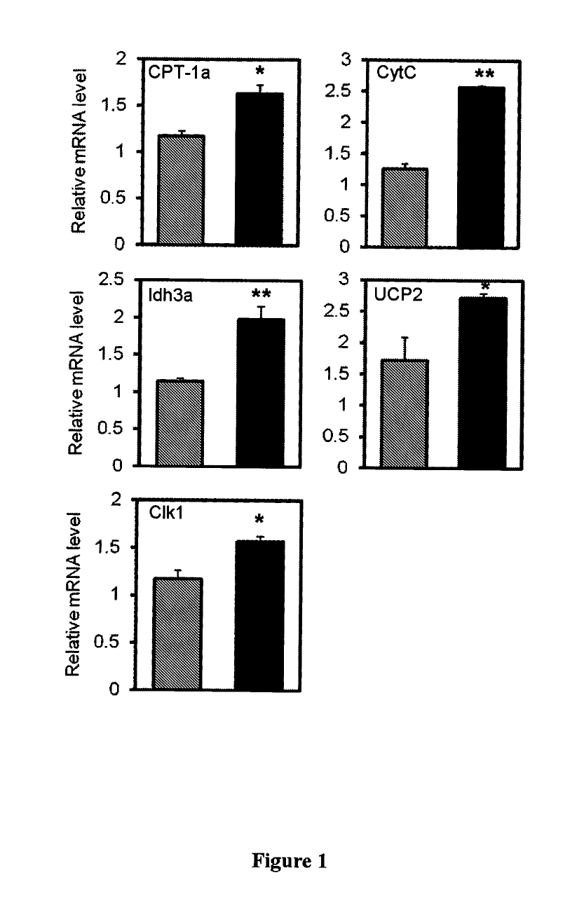

To determine if the effects of FGF21 are independent of body weight, we performed paired-feeding studies to match the body weight of vehicle-treated animals to FGF21-treated mice. In agreement with previous studies (11-14), non-fasted blood glucose were significantly decreased in FGF21-treated animals, but not in vehicle-treated or paired-fed animals (FIG. S2D). An oral glucose tolerance test revealed that FGF21 treatment significantly improved glucose tolerance (FIG. S2E) and decreased the area tinder the curve by 45% (FIG. S2F). Importantly, the levels of p-AMPK were increased in FGF21-treated animals by 53% but not in paired-fed animals (FIG. 1C). These results demonstrate that the effects of FGF21 on energy homeostasis and AMPK activation are independent of changes in body weight.

FGF21 increases NAD⁺ metabolism and SIRT1 activity. Recently, it has been demonstrated that AMPK indirectly activates SIRT1 by modulating intracellular NAD⁺/NADH levels. Since FGF21 increases AMPK activity, we sought to determine if FGF21 could alter intracellular NAD⁺/NADH ratios. Consistent with our hypothesis, FGF21 increased the NAD⁺/NADH ratio 48% (p<0.05, n=3) and 52% (p<0.05, n=3) over controls in 3T3-L1 and human adipocytes, respectively. Importantly, the NAD⁺/NADH ratio was increased by 40% (p<0.05, n=8) in white adipose tissue from ob/ob mice treated with FGF21 for 14 days, but not in paired-fed animals. Inhibition of AMPK activity with dominant-negative AMPK attenuated FGF21-stimulated increases in the NAD⁺/NADH ratio suggesting that AMPK activation by FGF21 is upstream of SIRT1 activation.

To determine whether the FGF21-induced elevation of NAD/NADH ratio could affect SIRT1 activity, we determined the acetylation status of the known SIRT1 substrate H3. Treatment of 3T3-L1, adipocytes with FGF21 decreased H3 acetylation by 48% (p<0.05, n=3), which was attenuated with shRNA knockdown of SIRT1. Furthermore, decreased acetylation of H3 (68%; p<0.05, n=8) was observed in white adipose tissues from FGF21-treated animals, but not in paired-fed mice. These data indicate that FGF21 alters NAD metabolism and activates SIRT1 activity in adipocytes in vitro and in vivo. Together, these results suggest that FGF21 controls energy metabolism through activation of AMPK and SIRT1.

FGF21 increases mitochondrial gene and protein expression. AMPK and SIRT1 are both critical regulators of mitochondrial biogenesis and function. To investigate the role of FGF21 in mitochondrial biogenesis and function, we analyzed the expression of genes involved in mitorhondrial function by gPCR in 3T3-L1 adipocytes, Treatment of 3T3-L1 adipocytes with FGF21 (4.0 μg/ml) for 3 days induced a significant increase in the expression of carnitine palmitoyltransferase 1A (CPT1a) by 1,6-fold, the enzyme involved in the transport of fatty acids across the mitochondrial membrane for fatty acid β-oxidation, isocitrate dehydrogenase 3 alpha (IDH3a) by 1.6-fold, responsible for the rate-limiting step of the tri-carboxylic acid (TCA) cycle, and cytochrome C (CytC) by 2.1-fold. Furthermore, these changes in gene expression correlated with increased levels of CytC protein by 6% (p<0.05, n=3).

Additionally, treatment of human adipocytes with FGF21 (4.0 μg/ml) significantly increased expression of genes involved in mitochondrial biogenesis and fatty acid β oxidation, including CPT1a (45%), PPARδ (23%), and PGC-1α(20%). These changes in gene expression also resulted in a significant increase in CytC protein levels by 13% (p<0.05, n=3).

FGF21 has been previously shown to regulate gene expression in vivo. Therefore, we examined mitochondrial protein expression in white adipose tissues from treated animals, CytC protein levels were elevated in FGF21 -treated animals by 32% (p<0.05, n=8), but not in vehide-treated or paired-fed mice.

FGF21 enhances mitoehondrial oxidative capacity. To further determine the effects of FGF21 on mitochondrial function, we measured mitochondrial enzymatic activity in 3T3-L1 treated with FGF21 (4.0 μg/ml) for 3 days by determining citrate synthase activity, a key component of the TCA cycle. FGF21 treatment of 3T3-L1 adipocytes significantly increased citrate synthase activity by 1.7-fold, suggesting that FGF21 enhanced TCA cycle activity,

To further assess the potential for FGF21 to increase mitochondrial oxidative capacity, we measured oxygen consumption in FGF21 treated 3T3-L1 and human adipocytes. FGF21 increased basal oxygen consumption by 1,2-fold (p<0.05, n=3), as well as oligomycin-treated oxygen consumption by 1,3-fold (p<0.05, n=3) in 3T3-L1 adipocytes. FCCP is a chemical uncoupler that abolishes the linkage between the respiratory chain and oxidative phosphorylation systems, and maximizes the respiratory capacity of mitochondria. FCCP-stimulated oxygen consumption was significantly increased by 1.9- and 1.7-fold (p<0.01, n=3), respectively, in 3T3-L1 and human adipocytes with FGF21 treatment, suggesting that FGF21 increases energy expenditure and enhances oxidative capacity.

AMPK and SIRT1 are required for FGF21-mediated effects on mitochondrial function. To determine if AMPK is required for FGF21-induced stimulation of mitochondrial function, We transduced human adipocytes with adenovirus over-expressing DN-AMPK. Inhibition of AMPK activity with dominant-negative AMPK attenuated FGF21 -stimulated increases in CPT-1a and PGC-1α gene expression and oxygen consumption.

Because AMPK activates SIRT1, we next evaluated whether SIRT1 mediates the effects of FGF21 on mitochondrial function downstream of AMPK. 3T3-L1 adipocytes were transduced with adenovirus either expressing SIRT1-shRNA or control shRNA. The cells were treated with FGF21 (4.0 μg/mL) for 3 days. SIRT1 mRNA levels were decreased by greater than 60% by the SIRT1-shRNA in both the PBS and FGF21-treated 3T3-L1 adipocytes. FGF21 treatment significantly increased oxygen consumption rates under basal (1.5-fold; p<0.05, n=3), as well as oligomycin- and FCCP-treatment (1.4-fold and 1.7-fold; p<0.05 and 0.01, n=3). The induction of cellular respiration by was abolished when SIRT1 was knocked down in these cells. Together, the data demonstrate that FGF21 enhances mitochondrial function via, an AMPK and SIRT1-dependent pathway.

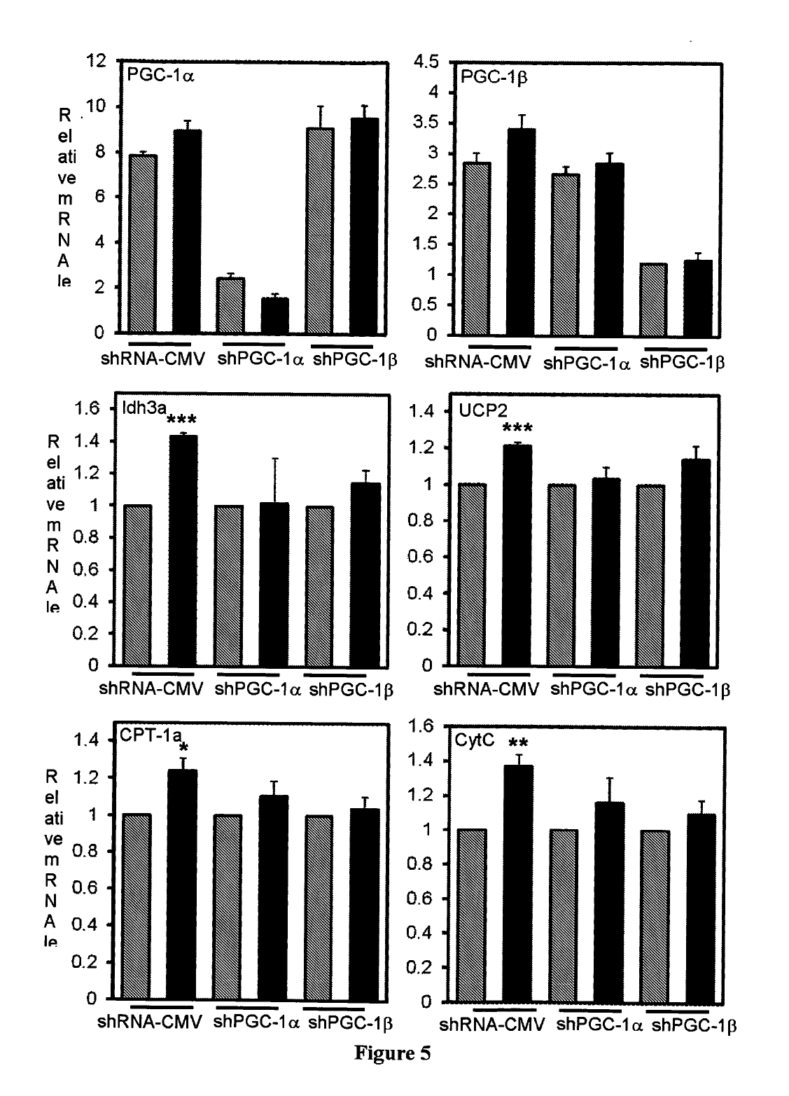

PGC-1α is required for FGF21-mediated effects on mitochondrial function. Since both AMPK and SIRT1 modulate PGC-1α expression and activity (22-24), we screened FGF21-treated 3T3-L1 adipocytes for increased expression of PGC-1α by qPCR. Interestingly, FGF21 did not appear to increase mRNA expression of PGC-1α in treated adipocytes (FIG. 5A). However, the activity of PGC-1α may be increased by FGF21 through post-translational modification, such as deaeetylation via SIRT1. Nevertheless, to determine if the regulation of mitochondrial function by FGF21 requires PGC-1α, we performed shRNA knockdown studies in 3T3-L1 adipocytes. PGC-1α mRNA levels were reduced by greater than 70% m 3T3-L1 adipocytes transduced with PGC-1α-shRNA adenovirus compared with controls. Knockdown of PGC-Lα attenuated the effects of FGF21 on CTP-1a and CytC gene expression and completely abolished FGF21-induced oxygen consumption at basal as well as with oligomycin and FCCP-treatment. Therefore, our results suggest that the coordinate effects of FGF21 on mitochondrial function require PGC-1α.

### Example 2

### Sequences

| FGF21 mRNA sequence (GenBank Accession No. NM_019113) (SEQ ID NO: 1): | |
|---|---|
| 1 | atggactcgg acgagaccgg gttcgagcac tcaggactgt gggtttctgt gctggctggt |
| 61 | cttctgctgg gagcctgcca ggcacacccc atccctgact ccagtcctct cctgcaattc |
| 121 | gggggccaag tccggcagcg gtacctctac acagatgatg cccagcagac agaagcccac |
| 181 | ctggagatca gagaggatgg gacggtgggg ggcgctgctg accagagccc cgaaagtctc |
| 241 | ctgcagctga aagccttgaa gccgggagtt attcaaatct tgggagtcaa gacatccagg |
| 301 | ttcctgtgcc agcggccaga tggggccctg tatggatcgc tccactttga ccctgaggcc |
| 361 | tgcagcttcc gggagctgct tcttgaggac ggatacaatg tttaccagtc cgaagcccac |
| 421 | ggcctcccgc tgcacctgcc agggaacaag tccccacacc gggaccctgc accccgagga |
| 481 | ccagctcgct tcctgccact accaggcctg ccccccgcac tcccggagcc acccggaatc |
| 541 | ctggcccccc agccccccga tgtgggctcc tcggaccctc tgagcatggt gggaccttcc |
| 601 | cagggccgaa gccccagcta cgcttcctga |
| FGF21 amino acid sequence (GenBank Accession No. NP_061986)(SEQ ID NO: 2): | |
| 1 | MDSDETGFEHSGLWVSVLAGLLLGACQAHPIPDSSPLLQFGGOVRQRYLY |
| 51 | TDDAQQTEAHLEIREDGTVGGAADQSPESLLQLKALKPGVIQILGVKTSR |
| 101 | FLCQRPDGALYGSLHFDPEACSFRELLLEDGYNVYQSEAHGLPLHLPGNK |
| 151 | SPHRDPAPRGPARFLPLPGLPPALPEPPGILAPQPPDVGSSDPLSMVGPS |
| 201 | QGRSPSYAS |

While the present invention has been described with reference to the specific embodiments thereof, it should be understood by those skilled in the art that various changes may be made and equivalents may be substituted without departing from the true spirit and scope of the invention. In addition, mans modifications may be made to adapt a particular situation, material, composition of matter, process, process step or steps, to the objective, spirit and scope of the present invention. All such modifications are intended to be within the scope of the present invention.

We claim:

1. A method of treating an AMPK-related cancer or cancer symptoms in a patient in need thereof comprising administering to the patient a theraputtically effective amount of FGF21 or an FGF21 agonist.

2. A method of assessing whether a subject afflicted with an AMPK-related cancer or cancer symptoms would be susceptible to treatment by FGF21 or FGF21 agonist administration comprising measuring AMPK or SIRT1 levels in a sample from said subject as compared to a control sample, wherein lower AMPK or SIRT1 levels in the subject sample compared to the control indicate that said subject would be amenable to treatment by FGF21 or FGF21 agonist administration.

3. A method of detecting a tumor in a patient afflicted with an AMPK-related cancer or cancer symptoms comprising administering to the patient a composition comprising FGF21 or an FGF21 agonist linked to an imaging agent and detecting the localization of the imaging agent in the patient.

4. A method of identifying an FGF21 agonist capable of AMPK-related cancer inhibition, comprising contacting a cell expressing AMPK with a candidate FGF21 agonist and determining whether AMPK levels are elevated relative to a control, whereby an observed elevation of AMPK levels indicates that said candidate FGF21 agonist is capable of AMPK-related cancer inhibition.
